(19) United States
(12) Patent Application Publication
(10) Pub. No.: US 2023/0167498 A1
(43) Pub. Date: Jun. 1, 2023

(54) METHOD OF DETERMINING THE PHENOTYPE OF A HAIR FOLLICLE

(71) Applicant: HairClone Limited, Manchester (GB)

(72) Inventors: Paul KEMP, Manchester (GB); Jennifer DILLON, Manchester (GB); Vincent RONFARD, Manchester (GB)

(73) Assignee: HairClone Limited, Manchester (GB)

(21) Appl. No.: 17/919,293

(22) PCT Filed: Apr. 16, 2021
(86) PCT No.: PCT/GB2021/050917

§ 371 Date: Oct. 17, 2022

(30) Foreign Application Priority Data
Apr. 16, 2020 (GB) 2005548.9

(51) Int. Cl. C12Q 1/6881

(52) U.S. Cl. CPC C12Q 1/6881; C12Q 2600/158; C12Q 2600/106

(57) ABSTRACT

The invention relates to methods of determining the phenotype of hair follicles. More specifically, the invention relates to a new method of determining the phenotype of a hair follicle by determining the expression profile of the cellular material attached to a plucked hair.

### FIELD OF THE INVENTION

The invention relates to methods of determining the phenotype of hair follicles. More specifically, the invention relates to a new method of determining the phenotype of a hair follicle by determining the expression profile of the cellular material attached to a plucked hair.

### BACKGROUND OF THE INVENTION

The treatment of hair loss is a significantly unmet public health need. Hair loss can have a serious impact on the self-confidence and consequent quality of life of an individual. It has also been shown recently that male-pattern baldness was significantly associated with increased risk of squamous cell carcinoma and basal cell carcinoma particularly in the scalp region (Li et al., 2016, Int J Cancer 139(12):2671-2678). Both males and females can suffer hair loss, with about 40% of men having noticeable hair loss by the age of 35 and about 80% of women experiencing noticeable hair loss by the age of 60. Androgenetic alopecia (AGA) is the most common form of hair loss in both men and women and is also known as male and female pattern baldness, respectively.

Hair grows from hair follicle structures which are multi-layered, angled invaginations of the superficial epithelium. The hair follicle unit is composed of both mesenchymal and epithelium-derived cell populations in close association. The germinative epithelial cells of the hair bulb proliferate and differentiate to give rise to the mature hair shaft. The mesenchyme component consists of fibroblast-like cells that form the morphologic units known as the dermal papilla (DP), located at the base of the hair follicle unit, and the dermal sheath (DS) that exists around the outer limits of the epithelial hair follicle component. The DP is essential to hair follicle development and cycling. Biochemical signaling by DP cells controls the cell dynamics of the epidermal component and the overall physical size of the hair follicle.

The hair has other structures such as nerves, sebaceous glands, a blood supply, and an attached arrector pili muscle that can alter the follicle's angle in the dermis. At the point at which this muscle attaches to the follicle itself there is a bulge region containing a reservoir of pluripotent epithelial stem cells called bulge cells.

Most body surfaces in humans, apart from soles of the feet, palms of the hand and lips contain hair follicles and it is estimated that an adult can have approximately 5 million hair follicles of which around 100,000 are on the scalp. The density of hair follicles varies with region from around 50 per cm² on the thigh to up to 600 per cm² on the scalp. As well as hair follicle density, hair shaft diameter and length are also very variable with fine vellus hairs approximately 10-30 μm in diameter covering the forehead, and nearby “terminal hairs” that are approximately 200 μm in diameter covering the scalp.

The development of a hair follicle begins when the basal cells of the epidermis form a visible hair placode and dermal fibroblasts begin to aggregate under the placode and differentiate to form a spherical DP. The epidermal cells form a multi-layered and elongated column called the hair peg which then thickens at the lower end to form a hair bulb which then encloses the elongated DP and the developing follicle grows down into the dermis. Differentiation of the epithelial cells in the hair peg into defined layers continues and a hair shaft is produced. Finally, this hair shaft protrudes from the skin surface and the hair follicle reaches its maximal length. The morphogenesis of a hair follicle thus requires intensive communications and joint development of the epidermal and dermal compartments.

Hairs show repeated cycles of growth and rest. The three stages of this cycle are the anagen, catagen, and telogen phases. Although in some animals, these cycles are synchronized, each strand of hair on the human body is asynchronous and at its own stage of this cycle. The anagen phase is the growth phase and can last from a few months to a few years. The longer the hair stays in the anagen phase, the longer it will grow. About 85% of the hairs on a human's scalp are in this anagen phase at any given time. This growth phase is then followed by the catagen phase which lasts about two weeks, during which time the cellular structure of the hair follicle degenerates to around ⅙ of its original length. While hair is not actively growing during this phase, the hair shaft is still anchored in place in the skin. Following catagen the hair follicle rests in the telogen phase in which the follicle remains dormant for one to four months. At some point, a new cycle is initiated and the follicle structure completely regenerates again. The hair base will break free from the root and this old hair shaft will be shed. Within two weeks a new hair shaft will begin to emerge as the next anagen phase begins. The new hair shaft is normally a similar size and structure to that produced in the previous cycle. In an average human life span a scalp hair will go through approximately 12 of these cycles. This cycling is known to be androgen dependent and changes in androgen levels can result in significant changes in hair shaft diameter and length in specific regions between one cycle and the next. During puberty for example changes in androgen levels can cause increase in hair shaft diameter in certain regions. However, androgens can also cause a reduction of hair shaft diameter and a miniaturization of the hair follicle itself through progressive cycles. It is this miniaturizing during conditions such as AGA which is most notable on the scalp.

Not all hair follicles respond to hormones in this way and AGA only affects certain androgen-sensitive hair follicles. On the scalp itself this is most notable in men as male pattern baldness, whereby hairs miniaturized in a progressive, temporal pattern beginning at the temples and spreading back across the scalp. This miniaturization of the diameter of hair follicles from terminal to vellus state occurs over a number of cycles. Moreover, during this miniaturizing process, the length of time of the hair follicle remains in the anagen or growth phase of the follicle decreases significantly, so the balding follicle spends a greater proportion of its follicle cycle in telogen, the non-growing stage and the hair shaft length as well as diameter decreases.

Hair follicle size and the resulting hair shaft diameter is determined by its mesenchymal component and it has been shown that human follicle DP miniaturization is a direct result of a reduction in papilla cell numbers in the hair follicle over time and it appears that this decrease occurs between anagen phases and generation of the next hair shaft.

Human DP cells from different body sites differ in their responsiveness to androgens. Particularly, androgens (for example, dihydrotestosterone [DHT] or 5α-dihydrotestosterone [5α-DHT]) stimulate hair follicle growth for example in the axilla, pubic area and male beard but cause follicle miniaturization in the scalp of patients suffering from AGA. Some hair follicles are insensitive to androgens and remain approximately the same size through life. Human DP cells can therefore be categorized as androgen inhibited (such as those affected by AGA) and androgen non-inhibited. These androgen non-inhibited DP cells can further be subdivided into androgen stimulated (such as in the male beard region) or androgen-insensitive (such as those hairs not affected by AGA).

Current hair loss therapies include oral and topical treatments and hair transplantation surgery. The currently available oral and topical treatments for hair loss are aimed at reducing the hair follicles sensitivity to androgens or extending the growth cycle. They must be taken life-long and are only effective during the early stages of hair loss. Only two therapeutic agents are currently approved by the US Food and Drug Administration (FDA) and European Medicines Agency (EMA) for the treatment of AGA: topical minoxidil and oral finasteride. Minoxidil can slow the progression of hair loss and partially restore hair but is expensive and effective in only a small proportion of males and females. Even for those individuals who respond, treatment must be maintained for life in order to sustain the effect. Minoxidil can also cause irritation and/or allergic reactions at the site of application. Finasteride is only effective in men, and then, only in a proportion of men. Finasteride may also cause long-term sexual side effects such as decreased libido and erectile dysfunction. Any beneficial effects on hair growth are lost within 6 to 12 months of discontinuing treatment.

Hair loss may alternatively be treated by transplanting hair follicles from regions that are expected to be unaffected by AGA to areas where hair loss is occurring (for example, frontal, mid scalp and crown region of the scalp). If the transplanted hairs have DP cells that are from mesodermal origin these will maintain a “positional memory” and continue to be unaffected by AGA thus providing a permanent solution. However, there are limitations on the numbers of follicles available for transplantation and the subject is left with the same number of hairs following transplantation, as the hair is merely distributed from high to low hair density regions. It is impossible therefore to restore a full head of hair.

Hair transplantation is the most cosmetically effective current treatment but, significantly, in men just beginning to show the effects of AGA, hair transplantation is often not suitable as the boundary of affected and unaffected regions are not yet clear and it is unknown both how many hair follicles will need to be transplanted and how many will be available. In women, hair transplantation is often not suitable as the pattern of hair loss is more diffuse and it is difficult to identify suitable donor sites. Using current techniques, it is therefore difficult for clinicians to identify regions containing hairs unaffected by AGA and hence suitable donor sites until AGA is advanced and hair loss visible. As a result, the effectiveness of hair transplantation is therefore limited by the patients who are suitable and the number of available donor hairs available. If hairs are transplanted before this boundary is known then it is possible that hairs with DP cells from neuro-ectodermal origin that are destined to be affected in the future are transplanted which would therefore only produce a temporary effect as the transplanted hair would miniaturize post-transplantation.

A 2015 survey of the International Society of Hair Restoration Surgeons (ISHRS), found that the most common complaint of hair transplant patients was either that the resultant hair density was less than expected (57% of all complaints) or that hair loss occurred post-transplant (39% of complaints). Hair transplantation is considered “hair restoration”, as it can only take place when the boundary of hair loss is properly understood in a particular patient. Consequently, approximately 80% of male transplant patients are over 30 years old and have already suffered significant hair loss.

Often subjects who would be willing to consider hair transplantation if available at an early stage (before visible/pronounced hair loss) no longer consider hair transplantation worthwhile if it only becomes clinically feasible once there is visible/pronounced hair loss. Despite the limits of this surgery, worldwide in 2016 there were over 500,000 surgical hair restoration procedures, creating a US $4.1 billion market. However, there is considerable potential to expand this market if screening for donor sites, and hence hair transplantation could be provided before AGA was advanced enough to cause visible/pronounced hair loss.

The present invention addresses these technical and clinical issues by providing a means to allow for earlier screening of hair follicles and identifying donor sites, and also to identify hair follicles suitable for treatment which have begun to miniaturize.

### SUMMARY OF THE INVENTION

The present inventors are the first to demonstrate that it is possible to classify hair follicles by determining the expression profile, particularly the transcriptome, of cellular material attached to the hair shaft. In particular, using a transcriptomics approach, the inventors have been able to classify hair follicles into those which are androgen-insensitive and hairs which are androgen-sensitive. Androgen-sensitive hair follicles are suitable as donor follicles for treatment (in the short-term or responsive to future hair loss). In addition, the inventors have been able to separate androgen-sensitive hair follicles into those which (i) are actively miniaturizing or have miniaturized; and (ii) those which are not currently miniaturizing but are destined to miniaturize in the future. This further classification allows for the identification of actively miniaturizing or miniaturized hair follicles which are suitable for treatment.

Accordingly, the present inventors provide for the first time a means to screen an individual to determine the phenotype, particularly androgen sensitivity of one or more individual hair follicles based on plucked hairs. This provides a simple, non-invasive method to obtain material for analysis. Furthermore, this method may be repeated one or more times to allow for repeated sampling from the same follicle. Androgen sensitivity resides in the DP cells and these remain behind when a hair is plucked (enabling the hair to regrow). However, as first appreciated by the present inventors, the expression profile of the adjacent epithelial cells (which remain attached to the plucked hair) is influenced by their DP cell counterparts and these changes provide an indirect indication of the current status, or phenotype, of the adjacent DP cell. Using this approach, hair follicles can be classified as androgen-insensitive or androgen-sensitive, with the androgen-sensitive follicles being further divided into those which would benefit from treatment in the immediate or short-term, and those which may benefit from treatment in the future. Thus, the method provided by the inventors allow the identification of one or more regions of the individual's scalp comprising hair follicles suitable for transplantation by determining the phenotype of hair follicles from said one or more regions using hair shafts plucked from said hair follicles. Further, this method has potential to predict regions of future hair loss and the extent thereof, and to identify suitable donor regions before the individual's hair loss becomes visible/pronounced.

Accordingly, the present invention provides an ex vivo method of determining the phenotype of a hair follicle comprising determining the expression profile of cellular material attached to a hair shaft plucked from said hair follicle.

In some embodiments the phenotype is androgen sensitivity.

The expression profile may be:
(a) a transcriptome or gene expression profile; or
(b) a protein expression profile.

The expression profile may be a transcriptome or gene expression profile determined by RT-qPCR, northern blotting, DNA/RNA microarray and/or RNA-Seq.

The expression profile may include the expression of one or more, preferably two or more, more preferably five or more of: NPNT, SPARC, LGR5, MGP, SLC47A1, PDPN, ITM2A, RERG, TNC, FAM171B, SORBS1, LOC105369434, LRRC15, PLA2G2F, HBA1, HBA2, C3orf52, PPP2R1B, ANKFN1, CACNA1C, LOC101929777 (WNT3), DST, GLUL, NTRK2, DIO2, SFRP1, LRIG1, CNTN4, COL17A1, TGFB2, IL31RA, SCRG1, ISM1, PAMR1, FGL2, WIF1, PCOLCE2, CDH11, DKK3, GAS1, FGF18, P2RY1, LINC00504, ALDH8A1, FAM26D, ADD2, FOSL1, HHIP, OSBPL1A, FHOD3, DMBT1, ZAR1, CTLA4, KIF5C, C10orf99, RNF152, SLC6A6, CRNDE, ANOS1 and FAM198B, or any combination thereof.

In some embodiments:
a) expression or up-regulation of one or more of NPNT, SPARC, LGR5, MGP, SLC47A1, PDPN, ITM2A, RERG, TNC, FAM171B, DST, IL31RA, SCRG1, ISM1, SFRP1, PAMR1, FLG2, WIF1, PCOLCE2, CDH11, NTRK2, DKK3, GAS1, FGF18, DKK3 and/or P2RY1, or any combination thereof is associated with androgen sensitivity; and/or
b) lack of expression or down-regulation of one or more of SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, DMBT1, ZAR1, CTLA4, KIF5C and/or C10orf99, or any combination thereof is associated with androgen sensitivity; and/or
c) expression or up-regulation of one or more of DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof is associated with androgen sensitivity.

In some embodiments:
a) expression or up-regulation of one or more of NPNT, SPARC, LGR5, MGP, SLC47A1, PDPN, ITM2A, RERG, TNC, and/or FAM171B, or any combination thereof is associated with androgen sensitivity;
b) lack of expression or down-regulation of one or more of SORBS1, LOC105369434, LRRC15, PLA2G2F, HBA1, HBA2, C3orf52, PPP2R1B, ANKFN1, CACNAI1 and/or LOC101929777 (WNT3), or any combination thereof is associated with androgen sensitivity; and/or
c) expression or up-regulation of one or more of DST, GLUL, NTRK2, FAM171B, DIO2, SFRP1, LRIG1, CNTN4, COL17A1, and/or TGFB2, or any combination thereof is associated with androgen sensitivity.

The invention also provides a method for assessing the extent of potential future hair loss in an individual, said method comprising carrying out a method as defined in any one of the preceding claims on hair shafts plucked from a plurality of hair follicles.

The boundary between androgen-sensitive and androgen-insensitive regions of an individual's scalp may be identified by said method, said method comprising the steps of:
(a) carrying out the ex vivo method of determining the phenotype of a hair follicle according to the invention on hair shafts plucked from one or more hair follicles from a region of the scalp of said individual;
(b) determining the sensitivity of said region to an androgen, wherein said region is androgen sensitive if said one or more hair follicles are androgen sensitive;
(c) repeating steps (a) and (b) one or more times on hair shafts plucked from one or more hair follicles from at least one different region of the scalp of said individual;
(d) generating a map of the androgen-sensitive and androgen-insensitive hair follicles within said regions of the scalp to identify the boundary between androgen-sensitive and androgen-insensitive regions of an individual's scalp.

The invention also provides a method for determining a region comprising hair follicles suitable:
(a) for hair transplantation in an individual; and/or
(b) as a source of cells for use in a method of rejuvenating hair follicles in an individual;

wherein said method comprises:
(i) carrying out the ex vivo method of determining the phenotype of a hair follicle according to the invention on hair shafts plucked from one or more hair follicles from a region of the scalp of said individual;
(ii) determining the sensitivity of said region to an androgen, wherein said region is androgen sensitive if said one or more hair follicles are androgen sensitive;
(iii) repeating steps (a) and (b) one or more times on hair shafts plucked from one or more hair follicles from at least one different region of the scalp of said individual;
(iv) generating a map of the androgen-sensitive and androgen-insensitive hair follicles within said regions of the scalp to identify the boundary between androgen-sensitive and androgen-insensitive regions of an individual's scalp;

wherein an area comprising hair follicles suitable for hair transplantation and/or as a source of cells for use in a method of rejuvenating hair follicles comprises one or more androgen-insensitive hair follicle.

Methods of the invention may be carried out before hair loss in said individual reaches visible levels, and preferably before hair loss commences.

The invention also provides a method for assessing the outcome of a previous hair follicle rejuvenation treatment, said method comprising carrying out the ex vivo method of determining the phenotype of a hair follicle according to the invention on hair shafts plucked from one or more hair follicles from a region previously containing androgen sensitive hair follicles and subjected to hair follicle rejuvenation; wherein the outcome is positive if there is a change in the expression profile of the cellular material attached to hair shafts plucked from said one or more hair follicles.

The change in the expression profile of the cellular material attached to the hair shafts plucked from said one or more hair follicles may be a change from an expression profile associated with androgen-sensitivity to an expression profile associated with androgen-insensitivity.

The expression profile associated with androgen-insensitivity may comprise:
a) lack of expression or down-regulation of one or more of NPNT, SPARC, LGR5, MGP, SLC47A1, PDPN, ITM2A, RERG, TNC, and/or FAM171B, or any combination thereof;
b) expression or up-regulation of one or more of SORBS1, LOC105369434, LRRC15, PLA2G2F, HBA1, HBA2, C3orf52, PPP2R1B, ANKFN1, CACNAI1 and/or LOC101929777 (WNT3), or any combination thereof; and/or
c) lack of expression or down-regulation of one or more of DST, GLUL, NTRK2, FAM171B, DIO2, SFRP1, LRIG1, CNTN4, COL17A1, and/or TGFB2, or any combination thereof.

The invention also provides a method for determining whether a hair follicle is miniaturizing and suitable for a hair rejuvenation treatment, said method comprising carrying out the ex vivo method of determining the phenotype of a hair follicle according to the invention, to determine the androgen-sensitivity of the hair follicle.

The expression profile associated with androgen-sensitivity may comprise:
a) expression or up-regulation of one or more of NPNT, SPARC, LGR5, MGP, SLC47A1, PDPN, ITM2A, RERG, TNC, and/or FAM171B, or any combination thereof;
b) lack of expression or down-regulation of one or more of SORBS1, LOC105369434, LRRC15, PLA2G2F, HBA1, HBA2, C3orf52, PPP2R1B, ANKFN1, CACNAI1 and/or LOC101929777 (WNT3), or any combination thereof; and/or
c) expression or up-regulation of one or more of DST, GLUL, NTRK2, FAM171B, DIO2, SFRP1, LRIG1, CNTN4, COL17A1, and/or TGFB2, or any combination thereof.

Hair loss may be due to androgenetic alopecia or the individual has androgenetic alopecia.

The androgen may be dihydrotestosterone (DHT).

In some embodiments, said upregulation and/or downregulation may be compared with the corresponding expression profile of cellular material attached to a hair shaft plucked from an androgen-insensitive hair follicle.

The hair shaft plucked from an androgen-insensitive hair follicle may be a hair shaft from the temporal or occipital region of the scalp, preferably a terminal hair shaft from the temporal region.

In some embodiments, said upregulation and/or downregulation may be compared with the corresponding expression profile of cellular material attached to a hair shaft plucked from an androgen-sensitive hair follicle.

The hair shaft plucked from an androgen-sensitive hair follicle may be a hair shaft from the frontal or vertex region of the scalp, preferably a minimizing or vellus hair shaft from the frontal region.

The individual may be a human and preferably the hair follicle is from the scalp.

The invention also provides a kit for collecting hair shafts for use in an ex vivo method of determining the androgen sensitivity of a plurality of hair follicles, the kit comprising:

a flexible cap defining:
(a) a receiving portion for accommodating a wearer's head, and
(b) a plurality of apertures through the flexible cap, the apertures distributed around the receiving portion and configured to allow a plurality of hair shafts from different regions of the wearer's scalp to be passed from the interior of the cap, through the aperture, to the exterior of the cap to allow hair shafts to be plucked from the hair follicles; and

a plurality of containers, wherein each container is labelled to correspond to a respective aperture of the plurality of apertures, for containing the plucked hair shafts from the different regions of the wearer's scalp.

Each aperture may be individually labelled with the same label as the corresponding labelled container.

The flexible cap may comprise markings for positioning the flexible cap with respect to the wearer's head such that each of the plurality of apertures are positioned over corresponding selected portion of the wearer's scalp for obtaining hair shafts from hair follicles from selected regions of the wearer's scalp.

### BRIEF DESCRIPTION OF THE DRAWINGS

FIG. 1: Representation of a subject wearing a flexible cap in accordance with the invention. The numbering indicates the plurality of apertures through which hair shafts can be passed.

FIG. 2: Representation of typical regions of the scalp as seen from the side (A) and top (B) of a subject's head.

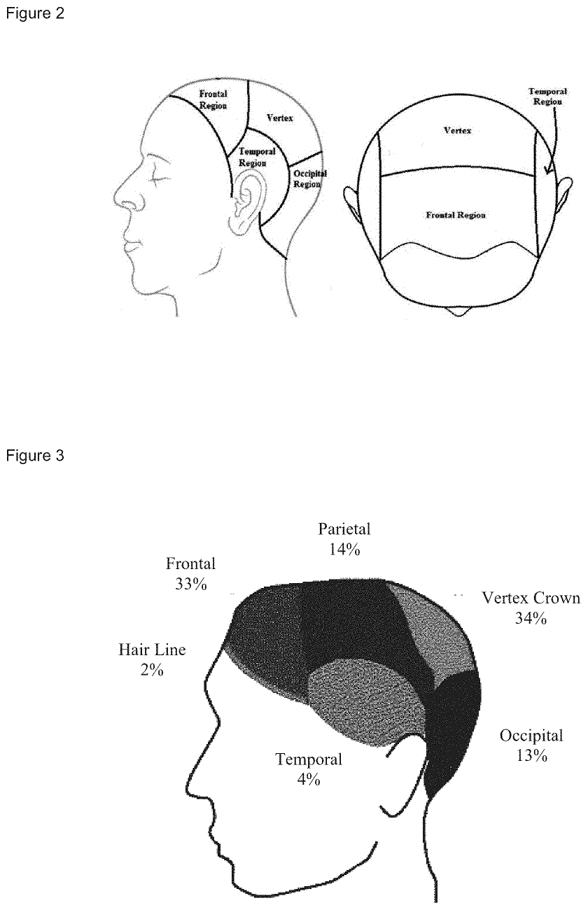

FIG. 3: Representation of six typical regions of the scalp as seen from the side of a subject's head.

FIG. 4: Image showing an individual undergoing a hair plucking procedure. As can be seen, the clinician exposes the area to be plucked by pulling up the adjacent hair.

FIG. 5: Representation of plucked hair shafts. Plucked hair shafts 1, 2 and 3 are particularly useful in methods of the invention.

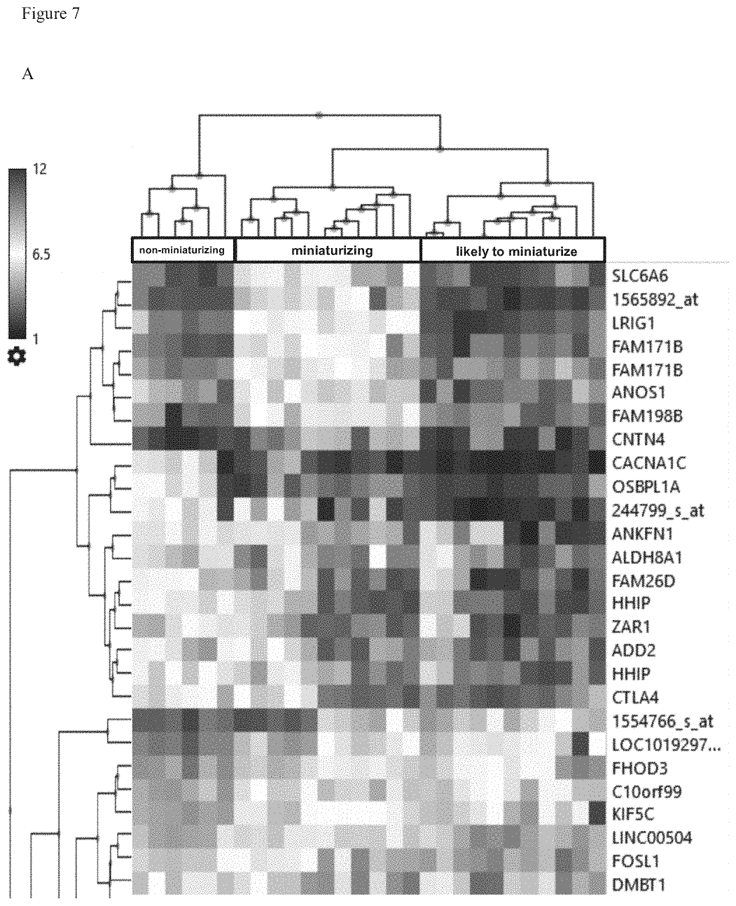

FIG. 6: Mosquito type tweezers.

FIG. 7: (A, B and C) Heat maps showing the differential gene expression of three distinct hair follicle phenotypes (non-miniaturizing, miniaturizing and likely to miniaturize), as determined by a method of the invention.

### DETAILED DESCRIPTION OF THE INVENTION

### Abbreviations

3D three-dimensional
5α-DHT 5-alpha-dihydrotestosterone
AGA androgenetic alopecia
DHT dihydrotestosterone
DP dermal papilla
EMA European Medicines Agency
FDA Food and Drug Administration
FUE follicle unit extraction
FUT follicular unit transplantation

### Terminology

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. This invention is not limited to the particular methodology, protocols, and reagents, etc., described herein and as such can vary. The terminology used herein is for the purpose of describing particular embodiments only and is not intended to limit the scope of the present invention, which is defined solely by the claims. Further, unless otherwise required by context, singular terms shall include pluralities and plural terms shall include the singular. In this application, the use of “or” means “and/or” unless stated otherwise. For example, “A and/or B” is to be taken as specific disclosure of each of (i) A, (ii) B and (iii) A and B, just as if each is set out individually. Furthermore, the use of the term “including”, as well as other forms, such as “includes” and “included”, is not limiting.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The various embodiments described herein can be combined to provide further embodiments. Aspects of the disclosure can be modified, if necessary, to employ the compositions, functions and concepts of the references herein and application to provide yet further embodiments of the disclosure. These and other changes can be made to the disclosure in light of the detailed description. All such modifications are intended to be included within the scope of the appended claims.

The technology illustratively described herein suitably may be practiced in the absence of any element(s) not specifically disclosed herein.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

A cosmetic method is defined as a method for improving the appearance of an individual.

Unless defined otherwise, all technical and scientific terms used herein have the same meaning as commonly understood by one of ordinary skill in the art to which this invention is related. Generally, nomenclatures utilized in connection with, and techniques of, cell and tissue culture, molecular biology, and protein and oligo- or polynucleotide chemistry and hybridization described herein are those well-known and commonly used in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

Standard techniques are typically used for tissue culture, including for proliferating cells in conditions suitable for proliferation. Suitable methods, reagents and condition for culturing cells obtained from hair follicle tissue are provided in the specific Examples. Enzymatic reactions and purification techniques may be performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to ±10% (and values there between, e.g., ±0.5%, ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%). The use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%).

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

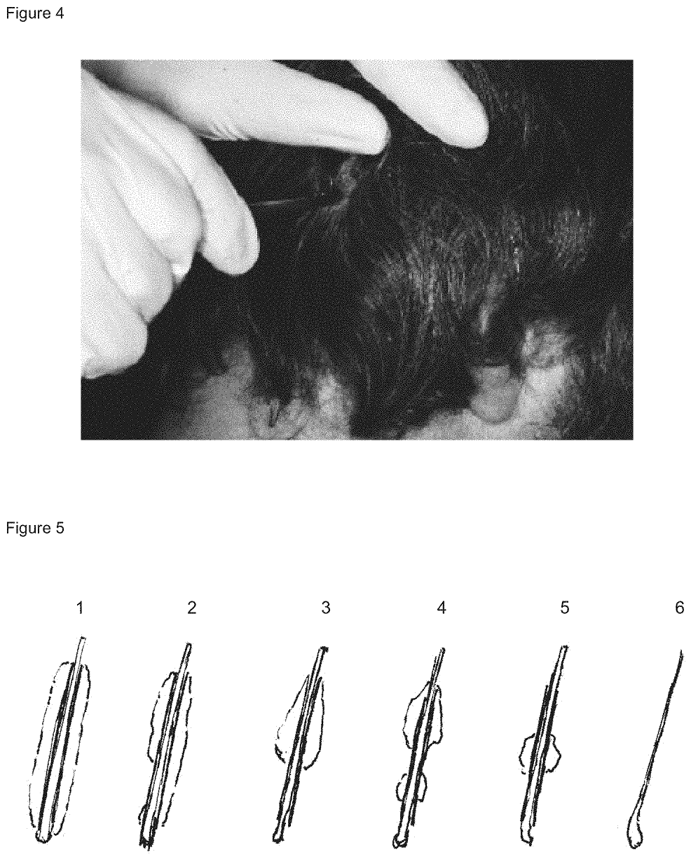

The term “region” as used herein refers to any location on the scalp of an individual. Typically, the term region refers to one of the six regions of the scalp as show in FIG. 3, i.e. the hair line, frontal, parietal, vertex (also known as the crown), occipital and temporal regions. The vertex and parietal regions may be referred to as a single region known as the vertex region.

Amino acids are referred to herein using the name of the amino acid, the three-letter abbreviation or the single letter abbreviation.

Unless otherwise indicated, any nucleic acid sequences are written left to right in 5′ to 3′ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide. For example, a fragment may comprise at least 5, at least 10, at least 11, at least 12, at least 13, at least 14, at least 15, at least 16, at least 17, at least 18, at least 19, at least 20 or more amino acids from a signal peptide of the invention. A fragment may be continuous or discontinuous, preferably continuous.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

As the skilled person will appreciate, and as described herein, a hair shaft is part of a hair follicle. In other words, a hair follicle comprises a hair shaft.

The entirety of each patent, patent application, publication and document referenced herein hereby is incorporated by reference to the same extent as if each individual publication, patent or patent application was specifically and individually indicated to be incorporated herein by reference.

The publications discussed herein are provided solely for their disclosure prior to the filing date of the present application. Nothing herein is to be construed as an admission that such publications constitute prior art to the claims appended hereto.

### Method of Determining the Phenotype of a Hair Follicle

The present inventors are the first to demonstrate that the phenotype of a hair follicle could be determined by determining the expression profile of the cellular material attached to a hair shaft simply plucked from said hair follicle. This is particularly surprising given that the cells attached to a plucked hair shaft (largely epithelial cells derived from the ectoderm) do not themselves direct the phenotype of the hair follicle. Instead, it is the mesenchymal cells of the dermal papilla (DP), and in particular the mesenchymal cells of the DP derived from the neuroectoderm which control production of the hair shaft and which determine the phenotype of the hair follicle.

Advantageously, therefore, the invention provides non-invasive methods for determining the phenotype of one or more hair follicle by using plucked hairs, as opposed to directly sampling the dermal papilla cells which requires invasive techniques (such as follicular unit extraction).

Accordingly, the invention provides a method of determining the phenotype of a hair follicle comprising determining the expression profile of cellular material attached to a hair shaft plucked from said hair follicle.

The method is typically carried out ex vivo. The hairs may be plucked by a medical or cosmetic practitioner, or by the individual. Plucking by the individual enables the individual to pluck one or more hair shafts for testing at a time and location convenient for them, and allows for home testing and kits for home testing (see below). The methods of the invention can be carried out subsequently, and at a distance from the site at which the hair shafts are plucked. Plucking also allows for repeated testing of a hair follicle over time.

As used herein, the term “phenotype” is intended to encompass the functional characteristics of the hair follicle. By way of non-limiting example, the expression profile of cellular material attached to a hair shaft may be indicate that the phenotype of the hair follicle is miniaturizing, non-miniaturizing or likely to miniaturize. The expression profile of cellular material attached to a hair shaft may indicate that the phenotype of the hair follicle is androgen-insensitive or androgen-sensitive.

A particular phenotype is typically associated with a particular gene expression profile as defined herein. As such, by determining the gene expression profile of the cellular material attached to a hair shaft plucked from a hair follicle, the phenotype of the hair follicle can be determined.

The present inventors have identified distinct expression profiles associated with different hair follicle phenotypes. Thus, advantageously, the methods of the invention are able to determine the phenotype of the hair follicle before any physical changes in the hair follicle or hair shaft can be observed (visually or microscopically).

### Methods of Determining Androgen Sensitivity

As described above, the present inventors have identified distinct expression profiles associated with different hair follicle phenotypes, and that these can be determined using the cellular material attached to a plucked hair shaft. Typically, the phenotype is androgen sensitivity. In other words, the method of the invention may be used to determine whether a hair follicle is sensitive or insensitive to an androgen (e.g. dihydrotestosterone (DHT), 5α-dihydrotestosterone (5α-DHT) and/or testosterone).

Accordingly, the invention provides a method of determining the androgen sensitivity of a hair follicle comprising determining the expression profile of cellular material attached to a hair shaft plucked from said hair follicle. Said methods may be used to determine whether a hair follicle is androgen-sensitive or androgen-insensitive.

The androgen-sensitivity of a hair follicle is typically linked to its miniaturization status or phenotype (the terms “miniaturization status” and “miniaturization phenotype” are used interchangeably herein). By miniaturization status, it is meant whether a hair follicle has undergone miniaturization, is currently undergoing miniaturization or is likely to undergo miniaturization in the future. For example, whether a hair follicle is actively miniaturizing or already miniaturized; not currently miniaturizing but likely/destined to miniaturize in the future; not currently miniaturizing and unlikely to undergo miniaturization in the future; or not miniaturized.

Androgen-sensitive hair follicles may be further categorized using the methods of the invention as (i) actively miniaturizing or already miniaturized; or (ii) not currently miniaturizing but are destined to miniaturize in the future. A miniaturizing hair follicle may be defined as a hair follicle in which there is a reduction in the number of DP cells in the hair follicle (typically compared with the number of DP cells in a non-miniaturizing or androgen-insensitive hair follicle). A miniaturizing hair follicle may also be identified by a reduction in diameter of the hair shaft compared to the hair shaft from a non-miniaturizing hair follicle. For example, the hair shaft in a hair follicle that is actively miniaturizing or already miniaturized may be less than about 0.06 mm (60 μm) in diameter, such as less than about 0.05 mm, less than about 0.04 mm or less than about 0.03 mm in diameter. Vellus hairs are hairs from miniaturized hair follicles, and are typically less than about 0.03 mm in diameter, for example 10-30 μm in diameter.

Androgen-insensitive hair follicles are typically non-miniaturizing. The term “non-miniaturizing” means that (i) the hair follicle has not miniaturized; (ii) is not currently miniaturizing; and/or (iii) is unlikely to undergo miniaturization in the future; or any combination of (i) to (iii) (e.g. (i) and (ii); (ii) and (iii), (i) and (iii); or (i), (ii) and (iii)). Preferably the term “non-miniaturizing” means that (i) the hair follicle has not miniaturized; (ii) is not currently miniaturizing; and (iii) is unlikely to undergo miniaturization in the future. Non-miniaturizing hair follicles typically contain terminal hairs. The hair shaft of an androgen-insensitive and/or non-miniaturizing follicle (e.g. terminal hair follicle) is typically at least about 0.06 mm (60 μm) in diameter, for example about 200 μm in diameter. These hair follicles are typically found in the occipital and temporal regions of the scalp.

As described herein, the inventors have identified three groups of genes/proteins that are useful in determining the androgen-sensitivity phenotype of a hair follicle:

Group 1 genes/proteins that are expressed or up-regulated in androgen-sensitive hair follicles (actively miniaturizing or already miniaturized; or not currently miniaturizing but are destined to miniaturize in the future), preferably up-regulated, compared with expression of the corresponding gene/protein in an androgen-insensitive hair follicle or non-miniaturizing hair follicle (not miniaturized; not currently miniaturizing; and/or unlikely to undergo miniaturization in the future).

Group 2 gene/proteins that are not expressed or are down-regulated in androgen-sensitive hair follicles (actively miniaturizing or already miniaturized; or not currently miniaturizing but are destined to miniaturize in the future), preferably down-regulated, compared with expression of the corresponding gene/protein in an androgen-insensitive hair follicle or non-miniaturizing hair follicle (not miniaturized; not currently miniaturizing; and/or unlikely to undergo miniaturization in the future).

Group 3 genes/proteins that are expressed or up-regulated in androgen-sensitive hair follicles (actively miniaturizing and/or already miniaturized), preferably up-regulated, compared with expression of the corresponding gene/protein in an androgen-sensitive hair follicle (not currently miniaturizing but are likely or destined to miniaturize in the future).

Using one or more (e.g. any two, three, four, five, six, seven, eight, nine, ten or more) gene/protein from any one of Groups 1, 2 and/or 3, it is possible to identify one or more hair follicle that is androgen-insensitive or androgen-sensitive. For example, one or more gene/protein from Group 1 (e.g. any two, three, four, five, six, seven, eight, nine, ten or more), one or more gene/protein from Group 2 (e.g. any two, three, four, five, six, seven, eight, nine, ten or more), or one or more gene/protein from Group 3 (e.g. any two, three, four, five, six, seven, eight, nine, ten or more) may be used. Two or more genes/proteins from Groups 1 and 2 (e.g. any two, three, four, five, six, seven, eight, nine, ten or more) may be used. Two or more genes/proteins from Groups 1 and 3 (e.g. any two, three, four, five, six, seven, eight, nine, ten or more) may be used. Two or more genes/proteins from Groups 2 and 3 (e.g. any two, three, four, five, six, seven, eight, nine, ten or more) may be used.

Using one or more (e.g. any two, three, four, five, six, seven, eight, nine, ten or more) gene/protein from Group 3, it is possible to identify one or more hair follicle that is androgen-sensitive and actively miniaturizing or already miniaturized and hence suitable for hair rejuvenation treatment.

Using one or more (e.g. any two, three, four, five, six, seven, eight, nine, ten or more) gene/protein from Groups 1 or 2, or Group 1 and 2 in combination allows for the differentiation of androgen-sensitive and androgen-insensitive hairs. This allows for the extent of future hair loss and/or the boundary between androgen-sensitive and androgen-insensitive hair follicles to be determined. This allows for treatment plans to be drawn up, and future donor regions and/or recipient regions to be identified. One or more gene/protein from Group 3 may be used in addition to one or more gene/protein from Group 1 and/or Group 2.

According to the invention, a hair follicle is androgen-sensitive if contact of the hair follicle with an androgen has a phenotypic effect on the hair follicle when compared to a relevant control (such as a hair follicle not contacted with the androgen or an androgen-insensitive follicle). Typically, this phenotypic effect is a reduction in the diameter of the hair shaft diameter and/or miniaturization of the hair follicle itself (as described herein). It has been shown that human follicle DP miniaturization is a direct result of reduction in papilla cell numbers in the hair follicle over time and it appears that this decrease occurs between anagen phases and generation of the next hair shaft.

According to the invention, the androgen-sensitivity of a hair follicle is typically determined indirectly, based on the expression profile of material attached to a hair shaft plucked from said follicle. Thus, according to the present invention a hair follicle is deemed androgen-sensitive if the material attached to the hair shaft plucked from said follicle shows an expression profile characteristic of androgen-sensitive regions. Alternatively, the profile could be analyzed directly on plucked hair follicle or after contacting the hair follicle with an androgen ex-vivo before analysis.

A hair follicle may be considered androgen-insensitive if contact of the hair follicle with an androgen has no phenotypic effect on the hair follicle when compared to a relevant control (such as a hair follicle not contacted with the androgen). Thus, androgen-insensitive hair follicles typically do not demonstrate miniaturization or a reduction in hair shaft diameter on contact with an androgen.

According to the invention, the androgen-insensitivity of a hair follicle is typically determined indirectly, based on the expression profile of material attached to a hair shaft plucked from said follicle. Thus, according to the present invention a hair follicle is deemed androgen-insensitive if the material attached to the hair shaft plucked from said follicle shows an expression profile characteristic of androgen-insensitive regions. Alternatively, the profile could be analyzed directly on a plucked hair follicle or after contacting the hair follicle with an androgen ex-vivo before analysis.

The androgen to which sensitivity is determined may be any androgen which is associated with hair loss or hair growth. In accordance with the invention, the androgen is a naturally occurring androgen or a synthetic androgen. Typically, the androgen is dihydrotestosterone (DHT), 5α-dihydrotestosterone (5α-DHT) and/or testosterone.

According to the invention, androgen sensitivity of a hair follicle is determined not by extracting and testing the hair follicle itself, but rather by plucking a hair shaft from said follicle. On plucking, cellular material from the follicle, remains attached to the hair shaft. It is this cellular material that is assayed according to the invention in order to determine the androgen sensitivity of the hair follicle. This cellular material is typically mainly comprised of epithelial cells which divide to produce the non-living hair shaft. These can be dead or live epithelial cells, as well as fragments thereof.

### Methods of Assessing the Extent of Potential Future Hair Loss

The invention further provides a method for assessing the extent of potential future hair loss in an individual, said method comprising carrying out the ex vivo method of determining the a phenotype of a hair follicle as described herein on hair shafts plucked from a plurality of hair follicles. Typically, the method comprises determining the androgen sensitivity of a hair follicle as described herein on hair shafts plucked from a plurality of hair follicles. The method may involve determining the miniaturization status of the one or more hair follicles, as described herein. Miniaturization status is typically linked with the androgen-sensitivity phenotype of a hair follicle, as described herein.

As used herein, an “individual” typically means a human. An “individual” in accordance with the invention can be male or female, adult or juvenile.

It will be appreciated that number of hair follicles to be assessed, and hence the number of hair shafts to be plucked may vary between individuals, for example based on the individual's family history of pattern baldness. Therefore, the number of hairs, and the regions from which they are plucked may be determined by a medical or cosmetic practitioner and adjusted, as necessary, to suit the individual.

Typically, given the preference for home testing or home sampling, a set number of hairs may be plucked from follicles at predefined positions within one or more specific region of the scalp. This predetermined sampling/plucking can be carried out by an individual in accordance with instructions provided. To this end, the invention further provides kits to assist an individual with plucking the desired hair shafts (see below).

In accordance with the invention, hair loss in an individual is also referred to as alopecia. Preferably, the hair loss is due to androgenetic alopecia. The term “androgenetic alopecia” as used herein is intended to encompass both male androgenetic alopecia and female androgenetic alopecia.

Methods of the invention can be used to determine the extent of potential future hair loss, the spatial distribution of potential future hair loss, or both, in an individual before hair loss commences. Extent of hair loss relates to the degree of hair follicle miniaturization within a region and can be readily determined using methods known in the art. The spatial distribution of hair loss relates to the regions of the scalp in which hair loss occurs. Again, appropriate methods and techniques for determining spatial distribution of hair loss are known in the art. Advantageously, this enables the individual to decide on whether any hair rejuvenation therapies (which involve the transfer of cellular material, e.g. DP cells) or hair transplant procedures will be necessary prior to the onset of visible sign of hair loss and the negative impacts on self-confidence and quality of life which may ensue. Furthermore, early determination of the extent and spatial distribution of potential future hair loss allows hair transplant specialists greater ability to accurately plan hair transplantation procedures, for example, by accurately determining suitable donor sites (i.e. regions comprising one or more androgen-insensitive hair follicles). Thus, typically a method of the invention is carried out before hair loss in an individual reaches visible levels. Preferably, methods of the invention are carried out before hair loss commences.

Hair loss may be quantified by any appropriate technique. Suitable techniques are known in the art, and can be practiced by one of ordinary skill. By way of non-limiting example, the Norwood scale can be used to measure male pattern baldness. The Norwood scale uses an illustrated representative of the progression of hair loss in men. The scale ranges from stages I to VII, with subdivisions within the scale. By way of another non-limiting example, the Ludwig scale can be used to measure baldness in women. This is similar to the Norwood scale in that it features multiple stages represented in diagrams or pictures. However, the Ludwig scale reflects the different pattern of hair loss progression in women compared with men. Other non-limiting examples of routine means to quantify hair loss include the hair mass index (HMI). For example, the methods of the invention may be carried out at stage 1, or before an individual reaches stage 2, 2A, 3 or 3A on the Norwood scale, or at stage I-1 or before an individual reaches stage I-2 or I-3 of the Ludwig scale.

Family history of baldness may be taken into account when deciding when to carry out the methods of the invention. For example, if an individual has a strong family history of pattern baldness, the methods are preferably carried out as earlier as possible (e.g. before hair loss commences or before Norwood stage 2 is reached), in view of the increased probability that the individual will also be affected over time.

The methods of the invention may also comprise identifying the boundary between different phenotypic regions of an individual's scalp. Accordingly, said method comprises the steps of: (a) carrying out a method of determining a phenotype of a hair follicle according to the invention on hair shafts plucked from one or more hair follicles from a region of the scalp of said individual; (b) repeating step (a) one or more times on hair shafts plucked from one or more hair follicles from at least one different region of the scalp of said individual; (c) generating a map of the different phenotypes of hair follicles within said regions of the scalp to identify the boundary between the different phenotypic regions of an individual's scalp.

In particular, the method may comprise identifying the boundary between androgen-sensitive and androgen-insensitive regions of an individual's scalp.

Accordingly, said method comprises the steps of: (a) carrying out a method of determining androgen sensitivity of a hair follicle according to the invention on hair shafts plucked from one or more hair follicles from a region of the scalp of said individual; (b) determining the sensitivity of said region to an androgen, wherein said region is androgen sensitive if said one or more hair follicles are androgen sensitive; (c) repeating steps (a) and (b) one or more times on hair shafts plucked from one or more hair follicles from at least one different region of the scalp of said individual; (d) generating a map of the androgen-sensitive and androgen-insensitive hair follicles within said regions of the scalp to identify the boundary between androgen-sensitive and androgen-insensitive regions of an individual's scalp. Any expression profile as described herein may be used to determine whether the one or more hair follicles is androgen-sensitive or androgen-insensitive.

The method of identifying the boundary between androgen-sensitive and androgen-insensitive regions of an individual's scalp may involve determining the miniaturization status of the one or more hair follicles, as described herein. Miniaturization status is typically linked with the androgen-sensitivity phenotype of a hair follicle, as described herein.

Accordingly, said method comprises the steps of: (a) carrying out a method of determining miniaturization status of a hair follicle according to the invention on hair shafts plucked from one or more hair follicles from a region of the scalp of said individual; (b) determining the miniaturization status of the one or more hair follicles, typically using an expression profile as described herein; (c) repeating steps (a) and (b) one or more times on hair shafts plucked from one or more hair follicles from at least one different region of the scalp of said individual; (d) generating a map of the miniaturization status of hair follicles within said regions of the scalp to identify the boundary between the different miniaturization status regions (e.g. currently undergoing miniaturization and not currently undergoing miniaturization; currently undergoing miniaturization and not currently undergoing miniaturization but likely/destined to undergo miniaturization in the future; currently undergoing miniaturization and not currently undergoing miniaturization and unlikely to undergo miniaturization in the future; not miniaturized and not currently undergoing miniaturization but likely/destined to undergo miniaturization in the future) of an individual's scalp. Any expression profile as described herein may be used to determine the miniaturization status of the one or more hair follicles.

### Methods for Determining Regions Comprising Hair Follicles Suitable for Transplantation

The invention provides a method for determining a region comprising hair follicles suitable for hair transplantation in an individual, wherein said method comprises: (a) carrying out a method of determining the phenotype of a hair follicle according to the invention, wherein said method is carried out on hair shafts plucked from one or more hair follicles from a region of the scalp of said individual; (b) repeating step (a) one or more times on hair shafts plucked from one or more hair follicles from at least one different region of the scalp of said individual; (d) generating a map of the different phenotypes of hair follicles within said regions of the scalp to identify the boundary between the different phenotypic regions of an individual's scalp; wherein an area comprising hair follicles suitable for hair transplantation comprises one or more hair follicle with a desired phenotype.

Typically the invention provides a method for determining a region comprising hair follicles suitable for hair transplantation in an individual, wherein said method comprises: (a) carrying out a method of determining androgen sensitivity of a hair follicle according to the invention, wherein said method is carried out on hair shafts plucked from one or more hair follicles from a region of the scalp of said individual; (b) determining the sensitivity of said region to an androgen, wherein said region is androgen sensitive if said one or more hair follicles are androgen sensitive; (c) repeating steps (a) and (b) one or more times on hair shafts plucked from one or more hair follicles from at least one different region of the scalp of said individual; (d) generating a map of the androgen-sensitive and androgen-insensitive hair follicles within said regions of the scalp to identify the boundary between androgen-sensitive and androgen-insensitive regions of an individual's scalp; wherein an area comprising hair follicles suitable for hair transplantation comprises one or more androgen-insensitive hair follicle. Any expression profile as described herein may be used to determine whether the one or more hair follicles is androgen-sensitive or androgen-insensitive.

The method for determining a region comprising hair follicles suitable for hair transplantation may involve determining the miniaturization status of the one or more hair follicles, as described herein. Miniaturization status is typically linked with the androgen-sensitivity phenotype of a hair follicle, as described herein.

Accordingly, said method comprises: (a) carrying out a method of determining the miniaturization status of a hair follicle according to the invention, wherein said method is carried out on hair shafts plucked from one or more hair follicles from a region of the scalp of said individual; (b) determining the miniaturization status of said region, typically using an expression profile as described herein; (c) repeating steps (a) and (b) one or more times on hair shafts plucked from one or more hair follicles from at least one different region of the scalp of said individual; (d) generating a map of the miniaturization status of hair follicles within said regions of the scalp to identify the boundary between the different miniaturization status regions of an individual's scalp; wherein an area comprising hair follicles suitable for hair transplantation comprises one or more hair follicle that is not currently miniaturizing; and unlikely to undergo miniaturization in the future and/or or not miniaturized. Any expression profile as described herein may be used to determine the miniaturization status of the one or more hair follicles.

As described above, typically such a method of the invention is carried out before hair loss in an individual reaches visible levels. Preferably, methods of the invention are carried out before hair loss commences.

Alternatively, methods of the invention may be carried out after hair loss has commenced, for example, after hair loss in an individual has reached visible levels. Methods of the invention may be carried out prior to a surgical or therapeutic intervention. By way of non-limiting example, methods of the invention may be carried out in an individual displaying visible signs of hair loss prior to undergoing hair transplantation surgery.

Whether the methods of the invention are carried out before hair loss commences, after commencement but prior to visible hair loss or after visible hair loss, the methods of the invention provide the clinician with accurate information about the extent and spatial distribution of potential future hair loss and allow the practitioner to accurately plan the hair transplantation procedure (or future hair transplantation procedures) by selecting follicles for transplantation from donor sites comprising androgen-insensitive follicles and not selecting follicles from regions with androgen-sensitive follicles for transplantation. Thus, the methods of the invention are advantageous, as they have the potential to reduce the risk of further hair loss after transplantation, and hence increase the probability of a positive outcome for the individual.

Typically, the invention relates to the identification of the boundary between androgen-sensitive and androgen-insensitive regions of the scalp, and to determining regions of the scalp comprising hair follicles suitable for hair transplantation. Therefore, preferably in the context of the invention, the androgen is DHT, 5α-DHT or DHT and 5α-DHT.

### Methods for Determining Regions Comprising Hair Follicles Suitable as a Source of Cells for Use in a Method of Rejuvenating Hair Follicles

The invention provides a method for determining a region comprising hair follicles suitable as a source of cells for use in a method of rejuvenating hair follicles in an individual, wherein said method comprises: (a) carrying out a method of determining a phenotype of a hair follicle according to the invention, wherein said method is carried out on hair shafts plucked from one or more hair follicles from a region of the scalp of said individual; (b) repeating step (a) one or more times on hair shafts plucked from one or more hair follicles from at least one different region of the scalp of said individual; (c) generating a map of the different phenotypic hair follicles within said regions of the scalp to identify the boundary between the different phenotypic regions of an individual's scalp, wherein an area comprising hair follicles suitable as a source of cells for use in a method of rejuvenating hair follicles comprises one or more hair follicle with a desired phenotype.

Typically, the invention provides a method for determining a region comprising hair follicles suitable as a source of cells for use in a method of rejuvenating hair follicles in an individual, wherein said method comprises: (a) carrying out a method of determining androgen sensitivity of a hair follicle according to the invention, wherein said method is carried out on hair shafts plucked from one or more hair follicles from a region of the scalp of said individual; (b) determining the sensitivity of said region to an androgen, wherein said region is androgen sensitive if said one or more hair follicles are androgen sensitive; (c) repeating steps (a) and (b) one or more times on hair shafts plucked from one or more hair follicles from at least one different region of the scalp of said individual; (d) generating a map of the androgen-sensitive and androgen-insensitive hair follicles within said regions of the scalp to identify the boundary between androgen-sensitive and androgen-insensitive regions of an individual's scalp; wherein an area comprising hair follicles suitable as a source of cells for use in a method of rejuvenating hair follicles comprises one or more androgen-insensitive hair follicle. Any expression profile as described herein may be used to determine whether the one or more hair follicles is androgen-sensitive or androgen-insensitive.

The method for determining a region comprising hair follicles suitable as a source of cells for use in a method of rejuvenating hair follicles in an individual may involve determining the miniaturization status of the one or more hair follicles, as described herein. Miniaturization status is typically linked with the androgen-sensitivity phenotype of a hair follicle, as described herein.

Accordingly, said method comprises: (a) carrying out a method of determining the miniaturization status of a hair follicle according to the invention, wherein said method is carried out on hair shafts plucked from one or more hair follicles from a region of the scalp of said individual; (b) determining the miniaturization status of said region, typically using an expression profile as described herein; (c) repeating steps (a) and (b) one or more times on hair shafts plucked from one or more hair follicles from at least one different region of the scalp of said individual; (d) generating a map of the miniaturization status of hair follicles within said regions of the scalp to identify the boundary between the different miniaturization status regions of an individual's scalp; wherein an area comprising hair follicles suitable as a source of cells for use in a method of rejuvenating hair follicles in an individual comprises one or more hair follicle that is not currently miniaturizing; and unlikely to undergo miniaturization in the future and/or or not miniaturized. Any expression profile as described herein may be used to determine the miniaturization status of the one or more hair follicles.

As used herein “hair rejuvenation therapy” comprises the transfer of cellular material (e.g. DP cell) from one or more androgen-insensitive hair follicle or hair follicle with an appropriate miniaturization status (e.g. that is not currently miniaturizing; and unlikely to undergo miniaturization in the future and/or or not miniaturized) to an androgen-sensitive or appropriate miniaturization status (activity miniaturizing and/or not currently miniaturizing; and likely/destined to undergo miniaturization in the future). Transfer of DP cells have been demonstrated to induce hair follicle development (e.g. McElwee et al. (2003) J. Invest. Dermatol. 121:1267-1275, which is herein incorporated by reference in its entirety). Therefore, identification of androgen-insensitive hair follicles and/or hair follicles with an appropriate miniaturization status (e.g. that is not currently miniaturizing; and unlikely to undergo miniaturization in the future and/or or not miniaturized) allows for DP cells from these follicles to be isolated and used for rejuvenation therapy.

As described above, typically such a method of the invention is carried out before hair loss in an individual reaches visible levels. Preferably, methods of the invention are carried out before hair loss commences. Suitable methods for quantifying hair loss are known in the art and non-limiting examples are described herein.

Alternatively, methods of the invention may be carried out after hair loss has commenced, for example, after hair loss in an individual has reached visible levels. Methods of the invention may be carried out prior to a surgical or therapeutic intervention. By way of non-limiting example, methods of the invention may be carried out in an individual displaying visible signs of hair loss prior to undergoing hair transplantation surgery.

Whether the methods of the invention are carried out before hair loss commences, after commencement but prior to visible hair loss or after visible hair loss, the methods of the invention provide the clinician with accurate information about the extent and spatial distribution of potential future hair loss and allow the practitioner to accurately plan the hair transplantation procedure (or future hair transplantation procedures) by selecting follicles for transplantation from donor sites comprising androgen-insensitive follicles and not selecting follicles from regions with androgen-sensitive follicles for transplantation. Thus, the methods of the invention are advantageous, as they have the potential to reduce the risk of further hair loss after transplantation, and hence increase the probability of a positive outcome for the individual.

Typically, the invention relates to the identification of the boundary between androgen-sensitive and androgen-insensitive regions of the scalp, and to determining regions of the scalp comprising hair follicles suitable for hair transplantation. Therefore, preferably in the context of the invention, the androgen is DHT, 5α-DHT or DHT and 5α-DHT.

### Methods for Determining Whether a Hair Follicle is Miniaturizing and Suitable for a Hair Rejuvenation Treatment

The invention also provides a method for determining whether a hair follicle is miniaturizing and suitable for a hair rejuvenation treatment, said method comprising carrying out the method of determining the phenotype of a hair follicle as described herein on hair shafts plucked from one or more hair follicles.

The method of the invention allows for the identification of hair follicles suitable for a hair rejuvenation treatment before hair loss in the individual reaches visible levels and preferably before hair loss commences. This is in contrast to current methods, which depend on a visual inspection of the hair shaft and follicles which is dependent on the skill of the practitioner and is time consuming.

Typically, the method comprises carrying out a method of determining the phenotype of a hair follicle as described herein on hair shafts plucked from one or more hair follicles, to determine the miniaturization status of the one or more hair follicle. Thus, said method may comprise determining whether the expression profile is indicative of a miniaturizing phenotype or likely to miniaturize phenotype (in other words, whether a hair follicle is actively miniaturizing or already miniaturized; not currently miniaturizing but destined to miniaturize in the future). If the expression profile of the cellular material attached to the plucked hair shaft is indicative of a miniaturizing or likely to miniaturize phenotype, the hair follicle from which the hair shaft was plucked may be considered suitable for a hair rejuvenation treatment.

Preferably, the method comprises carrying out a method of determining the phenotype of a hair follicle as described herein on hair shafts plucked from one or more hair follicles, to determine the androgen-sensitivity of the hair follicle. If the expression profile of the cellular material attached to the plucked hair shaft is indicative of androgen-sensitivity, the hair follicle from which the hair shaft was plucked may be considered suitable for a hair rejuvenation treatment.

Any appropriate expression profile may be used to determine the miniaturization status and/or androgen sensitivity of the one or more hair follicle. Suitable expression profiles are described herein.

### Methods for Assessing the Outcome of a Previous Hair Follicle Rejuvenation Treatment

The invention provides a method for assessing the outcome of a previous hair follicle rejuvenation treatment, said method comprising carrying out the ex vivo method of determining a phenotype of a hair follicle as described herein on hair shafts plucked from one or more hair follicles from a region previously containing hair follicles with a phenotype to be treated and subjected to hair follicle rejuvenation; wherein the outcome is positive if there is a change in the expression profile of the cellular material attached to hair shafts plucked from said one or more hair follicles (as described in detail below).

Typically, the invention also provides a method for assessing the outcome of a previous hair follicle rejuvenation treatment, said method comprising carrying out the ex vivo method of determining the androgen sensitivity of a hair follicle as described herein on hair shafts plucked from one or more hair follicles from a region previously containing androgen sensitive hair follicles and subjected to hair follicle rejuvenation; wherein the outcome is positive if there is a change in the expression profile of the cellular material attached to hair shafts plucked from said one or more hair follicles (as described in detail below). Thus, the invention allows for a medical or cosmetic practitioner to assess in an objective way the success of the previous hair follicle rejuvenation treatment.

The change in the expression profile of the cellular material attached to the hair shafts plucked from said one or more hair follicles may be a change from an expression profile associated with a miniaturizing phenotype to an expression profile associated with a non-miniaturizing phenotype. Thus, said method may comprise determining whether there has been a change from a miniaturizing phenotype or likely to miniaturize phenotype (in other words, whether a hair follicle is actively miniaturizing or already miniaturized; not currently miniaturizing but destined to miniaturize in the future) to a non-miniaturizing phenotype or not currently miniaturizing and unlikely to undergo miniaturization in the future.

Typically, the change in the expression profile of the cellular material attached to the hair shafts plucked from said one or more hair follicles is a change from an expression profile associated with androgen-sensitivity to an expression profile associated with androgen-insensitivity.

The expression profiles associated with a miniaturizing phenotype, a non-miniaturizing phenotype, androgen-sensitivity and androgen-insensitivity are provided herein.

The hair follicle rejuvenation treatment may, for example, use mesenchymal cells from androgen non-inhibited hair follicles. The hair follicle rejuvenation may, for example, be any hair rejuvenation treatment as described in WO 2020/079441 A1, which is hereby incorporated by reference.

Typically said method will be carried out after a sufficient period of time to allow the transplanted hair cells to become established in their new location. For example, said method can be carried out at least about three months, at least about six months, at least about nine months, at least about 12 months or at least about 18 months after the previous hair transplant or hair follicle rejuvenation treatment. Individual hair follicles could be identified by adjacent tattoo marks and the said method may be conducted multiple times over the course of the recovery period on the same follicles, to allow regular assessment of the viability of the treated follicles and the outcome of the previous hair follicle rejuvenation treatment. By way of non-limiting example, the method of the invention may be carried out at about 3 months, at about 6 months and at about 18 months after the previous hair transplant or hair follicle rejuvenation treatment. Additionally, this analysis could be repeated over multiple hair cycles in order to determine the long-term response of a rejuvenation treatment.

### Expression Profile

The term “change in the expression profile” as used herein is intended to encompass a change in the expression profile of the cellular material attached to hair shafts plucked from one or more hair follicles in a method of the invention. The methods of the invention are used to assess the expression profile of the cellular material attached to hair shafts plucked from one or more hair follicles to determine the phenotype of the hair follicle. Typically, the methods of the invention assess the expression profile of the cellular material attached to hair shafts plucked from one or more hair follicles to determine whether said hair follicles are androgen-sensitive or androgen-insensitive.

The methods of the invention may also be used to determine the miniaturization status of a hair follicle, or any change in miniaturization status. Changes in the expression profile of the cellular material attached to a hair shaft plucked from a hair follicle can also be used to determine whether said hair follicle has changed from a miniaturizing phenotype to non-miniaturizing phenotype, or from a non-miniaturizing phenotype to a miniaturizing phenotype. Changes in the expression profile of the cellular material attached to a hair shaft plucked from a hair follicle can also be used to determine whether said hair follicle has changed from an androgen-sensitive phenotype to an androgen-insensitive phenotype.

Accordingly, by determining the phenotype and/or androgen-sensitivity of hair follicles in a region previously subjected to hair follicle rejuvenation treatment, one can determine whether the rejuvenated hair follicle's expression profile has changed and will be prone to future hair loss (for example, due to androgen sensitivity), or whether they are non-miniaturizing and/or androgen-insensitive after treatment and have a greater chance of remaining.

By determining the expression profile of cellular material attached to a hair shaft plucked from a hair follicle, the phenotype and/or androgen sensitivity of said hair follicle can be determined. In accordance with the present invention, the expression profile may be a transcriptome, a gene expression profile, a protein expression profile, a micro RNA (miRNA) expression profile and/or a non-coding RNA (ncRNA) expression profile, or any combination thereof (for example, the expression profile may determine the expression of both genes and proteins). Preferably, the expression profile is a gene expression profile, more preferably a transcriptome expression profile.

A variety of means of determining gene expression are known to those skilled in the art. By way of non-limiting example, suitable methods to determine a transcriptome or gene expression profile in accordance with the present invention include real-time quantitative PCR (RT-qPCR), northern blotting, DNA/RNA microarray, RNA-Seq (also known as whole transcriptome shotgun sequencing) and/or serial analysis of gene expression (SAGE).

The inventors have identified numerous genes which are differentially expressed in the cellular material attached to hair shafts from androgen-sensitive hair follicles compared with the cellular material attached to hair shafts from androgen-insensitive hair follicles. These genes are also differentially expressed depending on the miniaturization status of a hair follicle. As such, one or more of these genes/proteins may be used to determine the androgen sensitivity and/or miniaturization status of a hair follicle according to the invention.

Accordingly, an expression profile of the invention may include the expression of one or more of NPNT, SPARC, LGR5, MGP, SLC47A1, PDPN, ITM2A, RERG, TNC, FAM171B, SORBS1, LOC105369434, LRRC15, PLA2G2F, HBA1, HBA2, C3orf52, PPP2R1B, ANKFN1, CACNA1C, LOC101929777 (WNT3), DST, GLUL, NTRK2, DIO2, SFRP1, LRIG1, CNTN4, COL17A1, TGFB2, IL31RA, SCRG1, ISM1, PAMR1, FGL2, WIF1, PCOLCE2, CDH11, DKK3, GAS1, FGF18, P2RY1, LINC00504, ALDH8A1, FAM26D, ADD2, FOSL1, HHIP, OSBPL1A, FHOD3, DMBT1, ZAR1, CTLA4, KIF5C, C10orf99, RNF152, SLC6A6, CRNDE, ANOS1 and FAM198B, or any combination thereof. Any two, three, four, five, six, seven, eight, nine, ten or more of these genes/proteins may form an expression profile according to the invention.

Exemplary sequences for each of these genes can be found in the indicated accession numbers (version numbers are indicated by the “.X” value), as accessed on 15 Apr. 2021: NPNT (NM_001184690.2), SPARC (NM_003118.4), LGR5 (NM_003667.4), MGP (NM_001190839.3), SLC47A1 (NM_018242.3), PDPN (NM_006474.5), ITM2A (NM_004867.5), RERG (NM_032918.3), TNC (NM_002160.4), FAM171B (NM_177454.4), SORBS1 (NM_006434.4), LOC105369434 [Is there another name for this gene? We cannot find it on NCBI], LRRC15 (NM_001135057.3), PLA2G2F (NM_022819.4), HBA1 (NM_000558.5), HBA2 (NM_000517.6), C3orf52 (NM_001171747.2), PPP2R1B (NM_002716.5), ANKFN1 (NM_001365758.1), CACNA1C (NM_199460.4), LOC101929777 (WNT3) (NM_030753.5), DST (NM_183380.4), GLUL (NM_001033044.4), NTRK2 (NM_006180.6), D102 (NM_013989.5), SFRP1 (NM_003012.5), LRIG1 (NM_015541.3), CNTN4 (NM_001206955.2), COL17A1 (NM_000494.4), TGFB2 (NM_001135599.4), IL31 RA (NM_139017.7), SCRG1 (NM_001329597.2), ISM1 (NM_080826.2), PAMR1 (NM_015430.4), FGL2 (NM_006682.3), WIF1 (NM_007191.5), PCOLCE2 (NM_013363.4), CDH11 (NM_001797.4), DKK3 (NM_001018057.2), GAS1 (NM_002048.3), FGF18 (NM_003862.3), P2RY1 (NM_002563.5), LINC00504 (NR_126435.1), ALDH8A1 (NM_022568.4), FAM26D (NM_001256887.3), ADD2 (NM_001185054.2), FOSL1 (NM_005438.5), HHIP (NM_022475.3), OSBPL1A (NM_018030.4), FHOD3 (NM_025135.5), DMBT1 (NM_004406.3), ZAR1 (NM_175619.3), CTLA4 (NM_005214.5), KIF5C (NM_004522.3), C10orf99 (NM_207373.3), RNF152 (NM_173557.3), SLC6A6 (NM_003043.6), CRNDE (NR_034105.4), and FAM198B (NM_001031700.3). Each of these sequences is herein incorporated by reference in their entirety. Variants and fragments of these genes/proteins (as described herein) are also encompassed.

As described above, these genes/proteins can be divided into three groups:

Group 1 genes/proteins that are expressed or up-regulated in androgen-sensitive hair follicles (actively miniaturizing or already miniaturized; or not currently miniaturizing but are destined to miniaturize in the future), preferably up-regulated, compared with expression of the corresponding gene/protein in an androgen-insensitive hair follicle or non-miniaturizing hair follicle (not miniaturized; not currently miniaturizing; and/or unlikely to undergo miniaturization in the future). Group 1 comprises or consists of NPNT, SPARC, LGR5, MGP, SLC47A1, PDPN, ITM2A, RERG, TNC, FAM171B, DST, IL31RA, SCRG1, ISM1, SFRP1, PAMR1, FLG2, WIF1, PCOLCE2, CDH11, NTRK2, DKK3, GAS1, FGF18, DKK3 and/or P2RY1, or any combination thereof. Group 1 may comprise or consist of NPNT, SPARC, LGR5, MGP, SLC47A1, PDPN, ITM2A, RERG, TNC, and/or FAM171B, or any combination thereof.

Group 2 gene/proteins that are not expressed or are down-regulated in androgen-sensitive hair follicles (actively miniaturizing or already miniaturized; or not currently miniaturizing but are destined to miniaturize in the future), preferably down-regulated, compared with expression of the corresponding gene/protein in an androgen-insensitive hair follicle or non-miniaturizing hair follicle (not miniaturized; not currently miniaturizing; and/or unlikely to undergo miniaturization in the future). Group 2 comprises or consists of SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, DMBT1, ZAR1, CTLA4, KIF5C and/or C10orf99, or any combination thereof. Group 2 may comprise or consist of SORBS1, LOC105369434, LRRC15, PLA2G2F, HBA1, HBA2, C3orf52, PPP2R1B, ANKFN1, CACNAI1 and/or LOC101929777 (WNT3), or any combination thereof.

Group 3 genes/proteins that are expressed or up-regulated in androgen-sensitive hair follicles (actively miniaturizing and/or already miniaturized), preferably up-regulated, compared with expression of the corresponding gene/protein in an androgen-sensitive hair follicle (not currently miniaturizing but are likely or destined to miniaturize in the future). Group 3 comprises or consists of DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof. Group 3 may comprise or consist of DST, GLUL, NTRK2, FAM171B, DIO2, SFRP1, LRIG1, CNTN4, COL17A1, and/or TGFB2, or any combination thereof.

Typically, the expression profile includes the expression of one or more gene/protein from Group 1, i.e. NPNT, SPARC, LGR5, MGP, SLC47A1, PDPN, ITM2A, RERG, TNC, FAM171B, PAMR1, SFRP1, ISM1, SCRG1, IL31RA, DST and/or DKK3, or any combination thereof.

Typically, the expression profile includes the expression of one or more gene/protein from Group 2, i.e. SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, HHIP, DMBT1, ZAR1, CTLA4, KIF5C, and/or C10orf99, or any combination thereof.

Typically, the expression profile includes the expression of one or more gene/protein from Group 3, i.e. DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof.

Preferably the expression profile may include the expression of: (a) one or more of NPNT, SPARC, LGR5, MGP, SLC47A1, PDPN, ITM2A, RERG, TNC, FAM171B, or any combination thereof; and/or (b) SORBS1, LOC105369434, LRRC15, PLA2G2F, HBA1, HBA2, C3orf52, PPP2R1B, ANKFN1, CACNAI1, WNT3, or any combination thereof; and/or (c) one or more of DST, GLUL, NTRK2, FAM171B, DIO2, SFRP1, LRIG1, CNTN4, COL17A1, and/or TGFB2, or any combination thereof.

Preferably, the expression profiles described herein include two or more, more preferably five or more of the genes/proteins listed herein.

One or more gene/protein may be selected independently from one or more of Groups 1, 2 and 3. For example an expression profile may include genes/proteins from one or more gene/protein from Group 1 only; one or more gene/protein from Group 2 only; one or more gene/protein from Group 3 only; one or more gene/protein from Groups 1 and one or more gene/protein from Group 2; one or more gene/protein from Group 1 and one or more gene/protein from Group 3; one or more gene/protein from Group 2 and one or more gene/protein from Group 3; or one or more gene/protein from each of Groups 1, 2 and 3.

The expression profile may further include the expression of one or more of DSP, SOX13, JAM3, SOAT1, NRARP, ATXN1L, TGFBR3, HISTH2AA, RFX3, PHACTR1, SHANK2, DIS3L2, ANK1, PARK2, NEDD4L, PLEKHH2, TMPO, ANKRD10, PROM1, NFIX, CD248, CRCP, EPHB1, TDRD1, IFF01, TIMP3, MMP25, MME, GABRB1, SLC39A5, LRRC26, KCNK5, B3GNT3, SLC26A4, CTH, ESRRG, PI16, APOBEC3D, CD244, KRT37, BMP5, DKK1, HOTAIR, HOXC9, CTNNAL, TGFB1, CHRM4, FGF5, NFATC2, HCRTR1, TPRX1, GRHL3, HOXC4, HOXC6, PRIMA1, MTR and/or PITX1, or any combination thereof.

Exemplary sequences for each of these genes can be found in the indicated accession numbers (version numbers are indicated by the “.X” value), as accessed on 15 Apr. 2021: DSP (NM_004415.4), SOX13 (XM_005245623.3), JAM3 (NM_032801.5), SOAT1 (NM_003101.6), NRARP (NM_001004354.3), ATXN1L (NM_001137675.4), TGFBR3 (NM_003243.5), HISTH2AA (NM_170745.3, RFX3 (NM_002919.4), PHACTR1 (NM_001242648.4), SHANK2 (NM_012309.5), DIS3L2 (NM_152383.5), ANK1 (NM_020476.3), PARK2 (NM_004562.3), NEDD4L (NM_001144967.3), PLEKHH2 (XM_017003351.2), TMPO (NM_003276.2), ANKRD10 (NM_017664.4), PROM1 (NM_006017.3), NFIX (NM_001271043.2), CD248 (NM_020404.3), CRCP (NM_014478.5), EPHB1 (NM_004441.5), TDRD1 (XM_011539962.1), IFF01 (NM_001039670.3), TIMP3 (NM_000362.5), MMP25 (XM_024450390.1), MME (NM_000902.5), GABRB1 (XM_024453977.1), SLC39A5 (NM_001135195.1), LRRC26 (NM_001013653.3), KCNK5 (NM_003740.4), B3GNT3 (NM_014256.4), SLC26A4 (NM_000441.2), CTH (NM_001902.6), ESRRG (NM_001438.4), PI16 (NM_001199159.2), APOBEC3D (NM_152426.4), CD244 (NM_016382.4), KRT37 (NM_003770.5), BMP5 (NM_021073.4), DKK1 (NM_012242.4), HOTAIR (NR_003716.3), HOXC9 (NM_006897.3), CTNNAL (NM_003798.4), TGFB1 (NM_000660.7), CHRM4 (NM_000741.5), FGF5 (NM_004464.4), NFATC2 (NM_001136021.3), HCRTR1 (NM_001525.3), TPRX1 (NM_198479.2), GRHL3 (NM_021180.4), HOXC4 (NM_014620.6), HOXC6 (NM_004503.4), PRIMA1 (NM_178013.4), MTR (NM_000254.3) and/or PITX1 (NM_002653.5). Each of these sequences is herein incorporated by reference in their entirety. Variants and fragments of these genes/proteins (as described herein) are also encompassed.

Androgen sensitivity may be determined by the expression or up-regulation of one or more genes and/or the lack of expression or down-regulation of one or more genes. The terms “up-regulation” and “down-regulation” are intended to encompass an increase or decrease respectively in the expression of a particular gene when compared to the expression of the same gene in cellular material derived from a hair shaft plucked from an androgen-insensitive hair follicle.

Typically, the expression or up-regulation of one or more of the Group 1 genes/proteins NPNT, SPARC, LGR5, MGP, SLC47A1, PDPN, ITM2A, RERG, TNC, FAM171B, PAMR1, SFRP1, ISM1, SCRG1, IL31RA, DST and/or DKK3, or any combination thereof is associated with a miniaturizing phenotype and/or androgen sensitivity. Conversely lack of expression or down-regulation of one or more of these genes/proteins may be associated with a non-miniaturizing phenotype and/or androgen insensitivity. Preferably, expression or up-regulation of one or more of the Group 1 genes/proteins NPNT, SPARC, LGR5, MGP, SLC47A1, PDPN, ITM2A, RERG, TNC, FAM171B, PAMR1, SFRP1, ISM1, SCRG1, IL31RA, DST and/or DKK3, or any combination thereof is associated with a hair follicle that has undergone miniaturization, is currently undergoing miniaturization or is likely to undergo miniaturization in the future. For example, a hair follicle is actively miniaturizing or already miniaturized; not currently miniaturizing but destined to miniaturize in the future. Preferably, lack of expression or down-regulation of one or more of the Group 1 genes/proteins NPNT, SPARC, LGR5, MGP, SLC47A1, PDPN, ITM2A, RERG, TNC, FAM171B, PAMR1, SFRP1, ISM1, SCRG1, IL31RA, DST and/or DKK3, or any combination thereof is associated with a hair follicle that is not currently miniaturizing and unlikely to undergo miniaturization in the future or is not miniaturized.

Typically, the lack of expression and/or down-regulation of one or more of the Group 2 genes/proteins SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, HHIP, DMBT1, ZAR1, CTLA4, KIF5C, and/or C10orf99, or any combination thereof is associated with a miniaturizing phenotype and/or androgen sensitivity. Conversely, expression or up-regulation of one of more these genes/proteins may be associated with a non-miniaturizing phenotype and/or androgen insensitivity. Preferably lack of expression or down-regulation of one or more of the Group 2 genes/proteins SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, HHIP, DMBT1, ZAR1, CTLA4, KIF5C, and/or C10orf99, or any combination thereof is associated with a hair follicle that has undergone miniaturization, is currently undergoing miniaturization or is likely to undergo miniaturization in the future. For example, a hair follicle that is actively miniaturizing or already miniaturized; or not currently miniaturizing but destined to miniaturize in the future. Preferably expression or up-regulation of one or more of the Group 2 genes/proteins SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, HHIP, DMBT1, ZAR1, CTLA4, KIF5C, and/or C10orf99, or any combination thereof is associated with a hair follicle that is not currently miniaturizing and unlikely to undergo miniaturization in the future or is not miniaturized.

Typically, the expression or up-regulation of one or more of the Group 3 genes/proteins DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof is associated with a miniaturizing phenotype and/or androgen sensitivity. Conversely lack of expression or down-regulation of one or more of these genes/proteins may be associated with a non-miniaturizing phenotype and/or androgen insensitivity. Preferably, expression or up-regulation of one or more of the Group 3 genes/proteins DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof is associated with a hair follicle that has undergone miniaturization or is currently undergoing miniaturization. For example, a hair follicle is actively miniaturizing or already miniaturized. Preferably, lack of expression or down-regulation of one or more of the Group 3 genes/proteins DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof is associated with a hair follicle that is not currently miniaturizing and unlikely to undergo miniaturization in the future or is not miniaturized; a hair follicle that is not currently miniaturizing but destined to miniaturize in the future, or a hair follicle that is not miniaturized.

Preferably, the expression or up-regulation of one or more of the Group 1 genes/proteins NPNT, SPARC, LGR5, MGP, SLC47A1, PDPN, ITM2A, RERG, TNC, and/or FAM171B, or any combination thereof is associated with a miniaturizing phenotype and/or androgen sensitivity. Conversely lack of expression or down-regulation of one or more of these genes/proteins may be associated with a non-miniaturizing phenotype and/or androgen insensitivity. Preferably, expression or up-regulation of one or more of the Group 1 genes/proteins NPNT, SPARC, LGR5, MGP, SLC47A1, PDPN, ITM2A, RERG, TNC, and/or FAM171B, or any combination thereof is associated with a hair follicle that has undergone miniaturization, is currently undergoing miniaturization or is likely to undergo miniaturization in the future. For example, a hair follicle is actively miniaturizing or already miniaturized; or not currently miniaturizing but destined to miniaturize in the future. Preferably, lack of expression or down-regulation of one or more of the Group 1 genes/proteins NPNT, SPARC, LGR5, MGP, SLC47A1, PDPN, ITM2A, RERG, TNC, and/or FAM171B, or any combination thereof is associated with a hair follicle that is not currently miniaturizing and unlikely to undergo miniaturization in the future or is not miniaturized.

Preferably, the lack of expression and/or down-regulation of one or more of the Group 2 genes/proteins SORBS1, LOC105369434, LRRC15, PLA2G2F, HBA1, HBA2, C3orf52, PPP2R1B, ANKFN1, CACNA1C, and/or LOC101929777 (WNT3), or any combination thereof is associated with a miniaturizing phenotype and/or androgen sensitivity. Conversely, expression or up-regulation of one of more these genes/proteins may be associated with a non-miniaturizing phenotype and/or androgen insensitivity. Preferably lack of expression or down-regulation of one or more of the Group 2 genes/proteins SORBS1, LOC105369434, LRRC15, PLA2G2F, HBA1, HBA2, C3orf52, PPP2R1B, ANKFN1, CACNA1C, and/or LOC101929777 (WNT3), or any combination thereof is associated with a hair follicle that has undergone miniaturization, is currently undergoing miniaturization or is likely to undergo miniaturization in the future. For example, a hair follicle is actively miniaturizing or already miniaturized; not currently miniaturizing but destined to miniaturize in the future. Preferably expression or up-regulation of one or more of the Group 2 genes/proteins SORBS1, LOC105369434, LRRC15, PLA2G2F, HBA1, HBA2, C3orf52, PPP2R1B, ANKFN1, CACNA1C, and/or LOC101929777 (WNT3), or any combination thereof is associated with a hair follicle that is not currently miniaturizing and unlikely to undergo miniaturization in the future or is not miniaturized.

Preferably, the expression or up-regulation of one or more of the Group 3 genes/proteins DST, GLUL, NTRK2, FAM171B, DIO2, SFRP1, LRIG1, CNTN4, COL17A1, and/or TGFB2, or any combination thereof is associated with a miniaturizing phenotype and/or androgen sensitivity. Conversely lack of expression or down-regulation of one or more of these genes/proteins may be associated with a non-miniaturizing phenotype and/or androgen insensitivity. Preferably, expression or up-regulation of one or more of the Group 3 genes/proteins DST, GLUL, NTRK2, FAM171B, DIO2, SFRP1, LRIG1, CNTN4, COL17A1, and/or TGFB2, or any combination thereof is associated with a hair follicle that has undergone miniaturization or is currently undergoing miniaturization. For example, a hair follicle is actively miniaturizing or already miniaturized. Preferably, lack of expression or down-regulation of one or more of the Group 3 genes/proteins DST, GLUL, NTRK2, FAM171B, DIO2, SFRP1, LRIG1, CNTN4, COL17A1, and/or TGFB2, or any combination thereof is associated with a hair follicle that is not currently miniaturizing and unlikely to undergo miniaturization in the future or is not miniaturized; a hair follicle that is not currently miniaturizing but destined to miniaturize in the future, or a hair follicle that is not miniaturized.

One or more genes/proteins from Group 3 as described herein may be used by themselves to identify hair follicles that are actively miniaturizing and thus suitable for hair rejuvenation therapy as described herein. In particular, expression or up-regulation of one or more genes/proteins from Group 3 as described herein may be used by themselves to identify hair follicles that are actively miniaturizing and thus suitable for hair rejuvenation therapy as described herein.

One or more genes/proteins from Group 1 and/or 2 as described herein may be used to assess the extent of potential future hair loss, identify the boundary between androgen-sensitive and androgen-insensitive regions of an individual's scalp, and/or determine a region comprising hair follicles suitable for hair transplantation or as a source of cells for use in a method of rejuvenating hair follicles as described herein. In some embodiments one or more genes/proteins from Group 3 as described herein may be used in addition to one or more gene/protein from Group 1 and/or 2 in such methods. In particular, lack of expression or down-regulation of one or more genes/proteins from Group 3 in combination with (i) expression or up-regulation of one or more genes/proteins from Group 1; and/or (ii) lack of expression or down-regulation of one or more genes/proteins from Group 2 may be used to identify hair follicles that are not currently miniaturizing but likely/destined to do so in the future.

The present inventors have also identified a number of genes and expression profiles which may be used to identify hair follicles which at present do not exhibit the visual characteristics of miniaturization, but which are likely to miniaturize based on their location, so called “likely to miniaturize” follicles (i.e. not currently miniaturizing but likely/destined to do so in the future). This same subset of hair follicles may at present be or appear to be androgen-insensitive but are likely to develop androgen-sensitivity.

This expression profile may be used to determine the extent of future hair loss.

The expression profile associated with hair follicles that are miniaturized or actively miniaturizing typically comprises expression or up-regulation of one of more of DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof. Lack of expression or down-regulation of one or more of DST, GLUL, NTRK2, FAM171B, DIO2, SFRP1, LRIG1, CNTN4, COL17A1, TGFB2, RNF152, SLC6A6, CRNDE, ANOS1 and/or FAM198B is typically associated with hair follicles that are not currently miniaturizing and unlikely to undergo miniaturization in the future; not miniaturized; or not currently miniaturizing but destined to miniaturize in the future.

Typically, an expression profile associated with hair follicles that are miniaturized or actively miniaturizing typically includes one or more of DST, GLUL, NTRK2, FAM171B, DIO2, SFRP1, LRIG1, CNTN4, COL17A1, TGFB2, RNF152, ANOS1, SLC6A6, FAM198B, and/or CRNDE, or any combination thereof. Preferably, an expression profile associated with hair follicles that are miniaturized or actively miniaturizing typically includes one or more of DST, GLUL, NTRK2, FAM171B, DIO2, SFRP1, LRIG1, CNTN4, COL17A1, and/or TGFB2 or any combination thereof.

An expression profile associated with hair follicles that could miniaturize (i.e. not currently miniaturizing but destined to miniaturize in the future) and/or develop androgen sensitivity may comprise the lack of expression or down-regulation of DST, GLUL, NTRK2, FAM171B, DIO2, SFRP1, LRIG1, CNTN4, COL17A1, TGFB2, RNF152, ANOS1, SLC6A6, FAM198B, and/or CRNDE, or any combination thereof. Said expression profile may include the expression or up-regulation of one or more gene/protein from Group 1 and/or the lack of expression and/or down-regulation of one or more gene/protein from Group 2.

Preferably, an expression profile associated with hair follicles that could miniaturize (i.e. not currently miniaturizing but destined to miniaturize in the future) and/or develop androgen sensitivity may comprises the lack of expression or down-regulation of DST, GLUL, NTRK2, FAM171B, DIO2, SFRP1, LRIG1, CNTN4, COL17A1, and/or TGFB2, or any combination thereof. Said expression profile may include the expression or up-regulation of one or more gene/protein from Group 1 and/or the lack of expression and/or down-regulation of one or more gene/protein from Group 2.

Up- and down-regulation may be compared to a suitable control. A control hair follicle may be taken from the same individual or a different individual. If from a different individual, the control individual may be matched for age, sex and/or other characteristics. Preferably the control hair follicle is from the same individual.

When used in relation to hair follicles that are miniaturizing and/or not currently miniaturizing but likely/destined to do so in the future, the terms “up-regulation” and “down-regulation” are intended to encompass an increase or decrease respectively in the expression of a particular gene when compared to the expression of the same gene in cellular material derived from a hair shaft plucked from a non-miniaturizing hair follicle.

When used in relation to hair follicles that are non-miniaturizing and/or not currently miniaturizing but unlikely to do so in the future, the terms “up-regulation” and “down-regulation” are intended to encompass an increase or decrease respectively in the expression of a particular gene when compared to the expression of the same gene in cellular material derived from a hair shaft plucked from a miniaturizing hair follicle and/or a hair follicle that is not currently miniaturizing but likely/destined to do so in the future.

When used in relation to androgen-sensitive hair follicles, the terms “up-regulation” and “down-regulation” are intended to encompass an increase or decrease respectively in the expression of a particular gene when compared to the expression of the same gene in cellular material derived from a hair shaft plucked from an androgen-insensitive hair follicle.

When used in relation to androgen-insensitive hair follicles, the terms “up-regulation” and “down-regulation” are intended to encompass an increase or decrease respectively in the expression of a particular gene when compared to the expression of the same gene in cellular material derived from a hair shaft plucked from an androgen-sensitive hair follicle.

As described herein, the skilled person can determine whether a hair follicle is non-miniaturizing and/or androgen-insensitive by well-established methods in the art, and therefore can readily obtain a hair shaft from such follicles to act as a control sample. Typically, hair follicles from the temporal or occipital regions of the scalp are non-miniaturizing and/or androgen-insensitive hair follicles. Preferably, a terminal hair shaft plucked from the temporal region of the scalp is used as a hair shaft plucked from a non-miniaturizing and/or androgen-insensitive hair follicle.

The term “down-regulation” or lack of expression when used in connection with an expression profile indicative of a hair follicle that could miniaturize and/or develop androgen sensitivity, is intended to encompass a decrease or absence of expression of a particular gene when compared to the expression of the same gene in cellular material derived from a hair shaft plucked from a miniaturizing hair follicle.

As described herein, the skilled person can determine whether a hair follicle is miniaturizing and/or androgen-sensitive by well-established methods in the art, and therefore can readily obtain a hair shaft from such follicles to act as a control sample. Typically, hair follicles from the frontal or vertex regions of the scalp are miniaturizing and/or androgen sensitive hair follicles. Preferably, a vellus hair shaft plucked from the frontal region of the scalp is used as a hair shaft plucked from a miniaturizing and/or androgen-sensitive hair follicle.

The expression or up-regulation of one or more of DSP, SOX13, JAM3, SOAT1, NRARP, ATXN1L, TGFBR3, HISTH2AA, RFX3, PHACTR1, SHANK2, DIS3L2, ANK1, PARK2, NEDD4L, PLEKHH2, TMPO, ANKRD10, PROM1, NFIX, CD248, CRCP, EPHB1, TDRD1, PfW1L4, IFF01, TIMP3, MMP25, MME, GABRB1, SLC39A5, LRRC26, KCNK5, B3GNT3, SLC26A4, CTH, MIDI, ESRRG, PI16, APOBEC3D and/or CD244, or any combination thereof is typically further associated with androgen sensitivity. Conversely down-regulation or lack of expression of one or more of these genes may be further associated with androgen insensitivity.

The down-regulation or lack of expression of one or more of KRT37, BMPS, DKK1, HOTAIR, HOXC9, CTNNAL, TGFB1, CHRM4, FGF5, NFATC2, HCRTR1, TPRX1, GRHL3, HOXC4, HOXC6, PRIMA1, MTR and/or PITX1, or any combination thereof may be further associated with androgen sensitivity. Conversely, up-regulation or expression of one or more of these genes may be further associated with androgen insensitivity.

In some embodiments: (a) the expression or up-regulation of one or more of DSP, SOX13, JAM3, SOAT1, NRARP, ATXN1L, TGFBR3, HISTH2AA, RFX3, PHACTR1, SHANK2, DIS3L2, ANK1, PARK2, NEDD4L, PLEKHH2, TMPO, ANKRD10, PROM1, NFIX, CD248, CRCP, EPHB1, TDRD1, PfW1L4, IFF01, TIMP3, MMP25, MME, GABRB1, SLC39A5, LRRC26, KCNK5, B3GNT3, SLC26A4, CTH, MIDI, ESRRG, PI16, APOBEC3D and/or CD244, or any combination thereof; and (b) down-regulation or lack of expression of one or more of KRT37, BMPS, DKK1, HOTAIR, HOXC9, CTNNAL, TGFB1, CHRM4, FGF5, NFATC2, HCRTR1, TPRX1, GRHL3, HOXC4, HOXC6, PRIMA1, MTR and/or PITX1, or any combination thereof is further associated with androgen sensitivity.

In some embodiments, the gene and/or protein expression profile does not include the androgen receptor (also known as NR3C4 (nuclear receptor subfamily 3, group C, member 4)).

The expression or non-expression of appropriate control genes known in the art (typically endogenous unregulated reference gene transcripts) may be included in the gene expression profile for normalization and/or comparative purposes.

In some embodiments, the expression profile is a protein expression profile, or the expression profile includes a protein expression profile. The protein expression profile may comprise the protein encoded by any one of the genes described herein, or any combination thereof. As such, all the disclosure above in relation to gene expression profiles and specific genes associated with androgen sensitivity or androgen insensitivity applies mutatis mutandis to the corresponding proteins and protein expression profiles.

A variety of means of determining protein expression are known to those skilled in the art. By way of non-limiting example, suitable methods to determine a protein expression profile in accordance with the present invention include enzyme-linked immunosorbent assay (ELISA), western blot, chromatography methods (for example, high-performance liquid chromatography) and/or mass spectrometry-based approaches.

Androgen sensitivity may be determined by the expression or up-regulation of one or more proteins and/or the down-regulation or lack of expression of one or more proteins. The terms “up-regulation” and “down-regulation” are intended to encompass an increase or decrease respectively in the expression of a particular protein when compared to the expression of the same protein in cellular material derived from a hair shaft plucked from an androgen-insensitive hair follicle.

The expression or non-expression of appropriate control proteins known in the art (typically proteins that exhibit high-level, constitutive expression in the cell type) may be included in the protein expression profile for normalization and/or comparative purposes.

A decrease or reduction in the expression of one or more gene and/or protein described herein encompasses a partial or total reduction. For example, expression may be reduced by at least 10%, at least 20%, at least 30%, at least 40%, at least 50%, at least 60, at least 70%, at least 80%, at least 90%, at least 95%, at least 99%, up to total elimination (knockout) of expression of the one or more gene and/or protein, typically compared with an appropriate control.

An increase in the expression of one or more gene and/or protein described herein encompasses an increase of at least 10%, at least 20%, at least 30%, at least 40%, at least 50%, at least 60, at least 70%, at least 80%, at least 90%, at least 100%, at least 150%, at least 200% compared with an appropriate control.

An increase in the expression of one or more gene and/or protein described herein may be expressed a fold change compared to expression of the same gene and/or protein in a control sample. The increase in expression may be at least 2-fold, 5-fold, 7-fold, 10-fold, 15-fold, 20-fold, 25-fold, 30-fold or 40-fold compared expression of the same gene and/or protein in the control sample.

A decrease in the expression of one or more gene and/or protein described herein may be expressed a fold change compared to expression of the same gene and/or protein in a control sample. The decrease in expression may be at least 2-fold, 5-fold, 7-fold, 10-fold, 15-fold, 20-fold, 25-fold, 30-fold or 40-fold compared expression of the same gene and/or protein in the control sample.

The expression level of the one/or more gene and/or protein as described herein encompasses the mass of the one/or more gene and/or protein, the molar amount of the one/or more gene and/or protein, the concentration of the one/or more gene and/or protein and the molarity of the one/or more gene and/or protein. This expression level may be given in any appropriate units. For example, the concentration of the one or more gene and/or protein may be given in pg/ml, ng/ml or μg/ml.

The expression level of the one/or more gene and/or protein as described herein may be measured directly or indirectly, for example using the non-limiting exemplary techniques described above.

### Kits

As discussed herein, one of the advantages of the present invention is that it allows for androgen-sensitivity of hair follicles to be determined based on the analysis of cellular material attached to hair shafts plucked from hair follicles, which allows for an individual to select (i.e. pluck) hair shafts for analysis themselves, rather than requiring a surgical procedure to extract follicles for direct testing. As such, the hair shafts may be plucked by the individual at a time and location of their choosing, such as home testing/sampling.

Accordingly, the invention provides a kit to facilitate the collection of appropriate numbers of hair shafts from predefined regions of the scalp to be tested by a practitioner subsequently (and at a different location).

The invention therefore provides a kit for collecting hair shafts for use in an ex vivo method of determining the androgen sensitivity of a plurality of hair follicles, the kit comprising: (a) a flexible cap defining: (i) a receiving portion for accommodating a wearer's head, and (ii) a plurality of apertures through the flexible cap, the apertures distributed around the receiving portion and configured to allow a plurality of hair shafts from different regions of the wearer's scalp to be passed from the interior of the cap, through the aperture, to the exterior of the cap to allow hair shafts to be plucked from the hair follicles; and (b) a plurality of containers, wherein each container is labelled to correspond to a respective aperture of the plurality of apertures, for containing the plucked hair shafts from the different regions of the wearer's scalp.

Each aperture of the flexible cap may be individually labelled with the same label as the corresponding labelled container to facilitate the correct identification of each of the plurality of hair shafts, enabling the practitioner carrying out the method of the invention to accurately identify the region from which each hair shaft was plucked.

The flexible cap may comprise markings for positioning the flexible cap with respect to the wearer's head such that each of the plurality of apertures are positioned over corresponding selected portion of the wearer's scalp for obtaining hair shafts from hair follicles from selected regions of the wearer's scalp. This helps to ensure that hair shafts are plucked from the predefined regions.

The present invention will now be described with reference to the following non-limiting Examples.

### EXAMPLES

### Example 1: Obtaining Plucked Hair Shafts

Before carrying out the hair shaft plucking protocol, it is preferable to ensure that the patient has not washed their hair for at least 24 hours prior to plucking, as shampoo and other hair care products may interfere with sample quality.

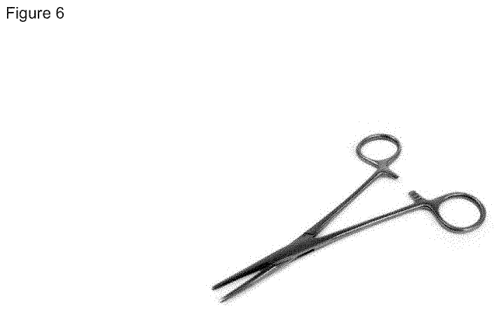

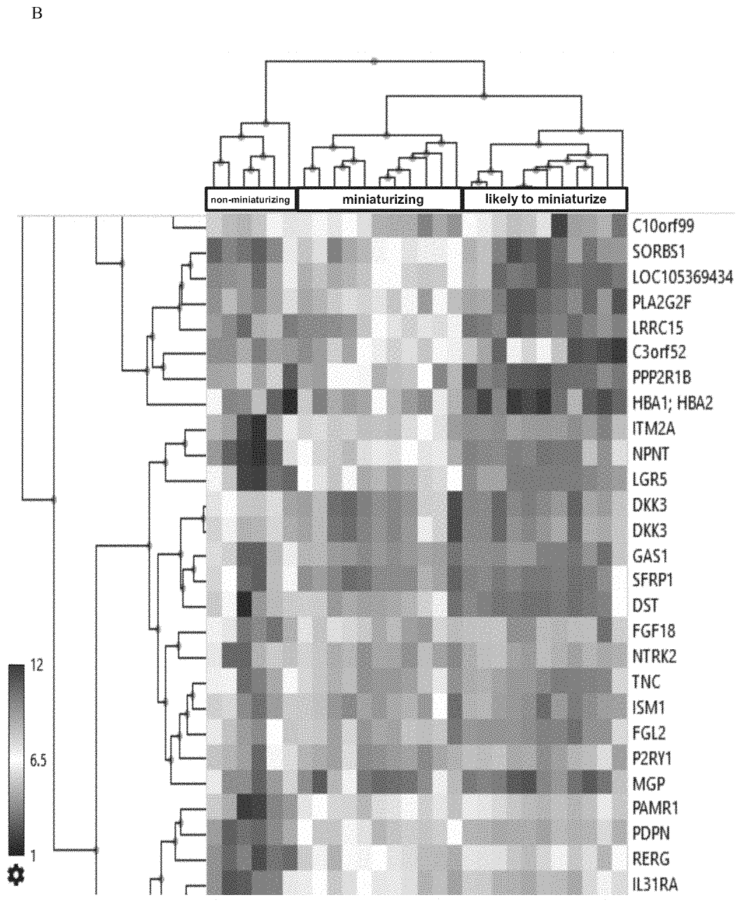

The protocol begins by first exposing the area to be plucked by pulling up the adjacent hair (as show in FIG. 4). Next, using tweezers (mosquito type, as shown in FIG. 6) anywhere from between 1 to 30 hair shafts are gripped as close to the surface of the skin as possible and clamped in the tweezers.

The clamped hair is then pulled out as rapidly as possible using a jerky, or sudden, movement in a direction co-axial to hair growth. This strategy ensures that the root sheath is harvested together with the hair shaft. The hair shafts should then be placed in a dish containing phosphate buffered saline before isolating the mRNA for analysis.

### Example 2: Isolating mRNA for Analysis

RNA from the hair shafts obtained in Example 1 is extracted using the RNeasy Plus Micro Kit (Qiagen). RNA is used to synthesize first-strand complementary DNA (cDNA) using Nugen Ovation V2. This is converted to double-stranded cDNA, and used as a template for in vitro transcription to generate cRNA using the Nugen Encore Biotin Module. The cRNA is then transferred for hybridization and scanning using an Affymetrix U133 Plus 2.0 Array (ThermoFisher Scientific Catalog no. 900466). Differential gene expression analysis is performed using Transcriptome Analysis Console (TAC) software (ThermoFisher Scientific). Expression or non-expression of appropriate control genes known in the art are tested for comparative purposes.

### Example 3: Isolating Protein for Analysis

Hair shaft protein is extracted by homogenising the plucked hair shaft (or a portion thereof) in radioimmunoprecipitation buffer (RIPA, Thermo Fisher Scientific) containing complete protease inhibitor cocktail (Roche) and phosphatase inhibitor cocktail 2 (Sigma Aldrich).

Protein expression is then determined by any appropriate method, for example, western blot or ELISA.

Protein expression of the hair shaft is also determined using a tandem mass spectrometry approach.

### Example 4: Determining Potential Androgen Sensitive Regions of a Patient's Scalp

Hair shafts are plucked in accordance with example 1, from a region in the hair line directly above the nose at the front to the scalp. Genetic analysis is carried out according to example 2. Using the genes described herein it is possible to discriminate between androgen non-inhibited, hair follicle inductive cells and androgen-sensitive cells. Expression or non-expression of appropriate control genes known in the art are be tested for comparative purposes.

### Example 5: Using a Kit to Determine Androgen Sensitive Regions of Patient's Scalp

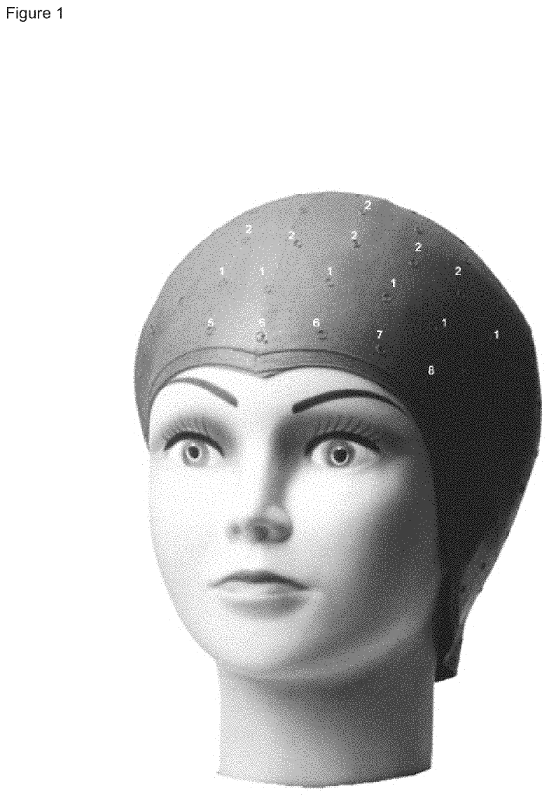

A flexible cap with a series of holes as shown in FIG. 1 is placed over the patient's head. The cap is positioned with the V shaped portion over the bridge of the nose and the edges of the cap at the height of the eyebrows. Approximately 10 hairs are pulled through the holes and plucked according to example 1. Their location on the cap is noted and RNA isolated from the cellular material surrounding the hair and analyzed according to example 2.

### Example 6: Determining the Boundary Between Androgen Sensitive and Insensitive Regions

The expression profiles of hairs obtained and analyzed according to example 5 can be compared across different sites on an individual's scalp to produce a “map” of expected androgen sensitivity.

This map is used to inform the clinician of the boundaries between androgen sensitive and insensitive regions and thus help determine the most effective treatment protocol for the individual.

### Example 7: Determining Response of Androgen Sensitive Follicles to Rejuvenation Treatment

Six months following a hair follicle rejuvenation treatment, hair follicles having been subject to a rejuvenation treatment are analyzed to determine their androgen sensitivity, and thus whether the treatment was a success.

Hair shafts from previously rejuvenated areas of the scalp are plucked according to example 1. RNA is isolated and analyzed according to example 2.

The analysis reveals that the hair follicles are androgen insensitive and therefore that the rejuvenation treatment has been successful.

### Example 8: Transcriptomic Analyses of the Cellular Material Surrounding Hair Shafts

A transcriptomics-based approach was used to determine the expression profile of the cellular material attached to different hair shaft types.

Three different types of hair shaft were obtained from individuals:

(1) hair shafts unlikely to miniaturize with wide hair shaft diameters (from the occipital region of men and women; N=9, five male and four female);
(2) hair that has miniaturized or is miniaturizing with smaller hair shaft diameters (from the frontal region in men and temporal region in women; N=9, five male and four female);
(3) hair that has the appearance of non-miniaturized hair but is likely to undergo miniaturized in the future (N=10, five individuals, two samples from each).

Using the method described in example 1, hair was plucked by gripping a group of approximately 5-10 hairs close to the root and pulling quickly to remove epithelial cell attached to the hair shaft. A total of 15-20 hairs were plucked and placed into a 2 mL tube of RNAlater and stored at room temperature or 4° C. for up to five days until RNA extraction was carried out. For some samples, once transported in RNAlater, hair was washed in PBS then frozen at −80° C. until RNA extraction could be carried out.

RNA was extracted from hairs using the Qiagen RNeasy Plus Micro Kit as described in Example 2 and eluted into nuclease-free water. Samples were stored at −80° C. and shipped on dry ice to the Bioinformatics facility (Bioinformatics UK, Woldingham).

RNA was assessed for integrity then run on an Affymetrix GeneChip® Human Genome U133 Plus 2.0 Array.

Results were analyzed using the Affymetrix Transcription Analysis Console (TAC) to determine which gene were most differentially regulated between non-miniaturizing and miniaturizing plucked hairs.

Genes were filtered by those with 5-fold expression differences between the miniaturizing and non-miniaturizing samples and gene with a false discovery rate (FDR) F-test value below 0.05.

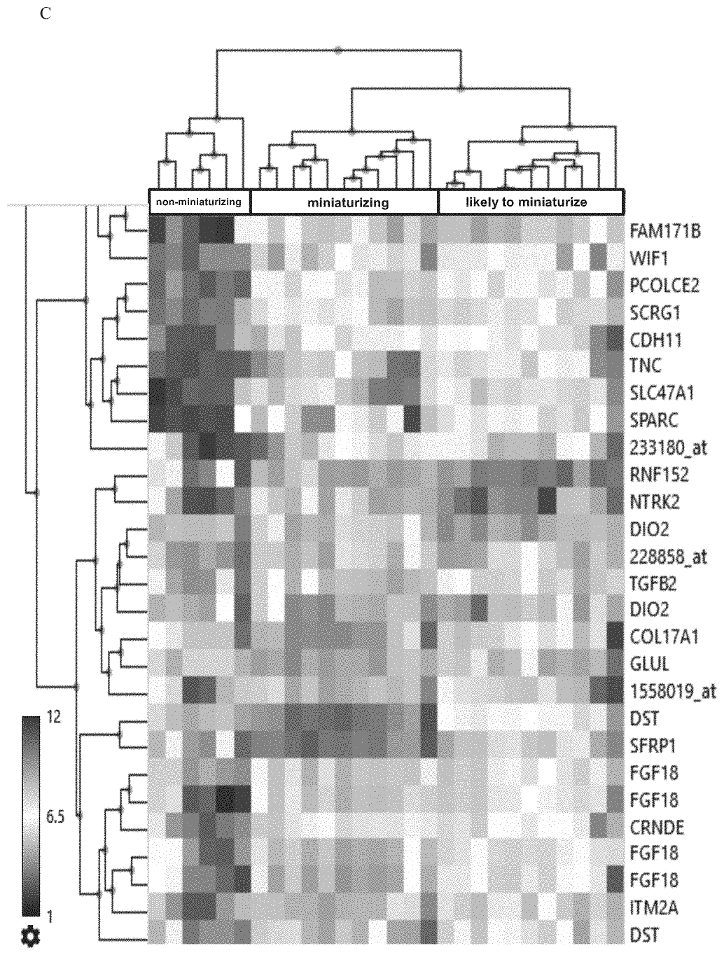

Expression levels of the various genes was visualized in a heatmap as shown in FIGS. 7A, B and C. The raw data is given in Tables 1 to 3.

Analysis of the results revealed three different groups of genes: Group 1 (up-regulated in miniaturized/destined to miniaturize compared with thick/non-miniaturized hairs); Group 2 (down-regulated in miniaturized/destined to miniaturize compared with non-miniaturizing hairs); and Group 3 (up-regulated in miniaturized vs destined to miniaturize/non-miniaturizing hairs). The top 10 genes identified in each group are as follows:

Group 1 NPNT, SPARC, LGR5, MGP, SLC47A1, PDPN, ITM2A, RERG, TNC, and/or FAM171B
Group 2 SORBS1, LOC105369434, LRRC15, PLA2G2F, HBA1 and HBA2, C3orf52, PPP2R1B, ANKFN1, CACNAI1 and/or LOC101929777 (WNT3)
Group 3 DST, GLUL, NTRK2, FAM171B, DIO2, SFRP1, LRIG1, CNTN4, COL17A1, and/or TGFB2

TABLE 1
Upregulated genes in actively miniaturizing/not currently miniaturizing
but likely to miniaturize vs. non-miniaturizing hair follicles.

| | | | | Likely to |
|---|---|---|---|---|
| | | | Miniaturizing | miniaturize |
| | | | vs. non- | vs. non- |
| | Gene | | miniaturizing | miniaturizing |
| Transcript ID | Symbol | Gene Name | (Fold Change) | (Fold Change) |
| 202747_s_at | ITM2A | integral membrane protein | 9.31 | 4.17 |
| | | 2A | | |
| 231925_at | P2RY1 | purinergic receptor P2Y, G- | 5.43 | 4.49 |
| | | protein coupled, 1 | | |
| 231382_at | FGF18 | fibroblast growth factor 18 | 5.78 | 6.3 |
| 204457_s_at | GAS1 | growth arrest-specific 1 | 5.07 | 6.34 |
| 221796_at | NTRK2 | neurotrophic tyrosine | 7.58 | 7.17 |
| | | kinase, receptor, type 2 | | |
| 207173_x_at | CDH11 | cadherin 11, type 2, OB- | 5.13 | 7.28 |
| | | cadherin (osteoblast) | | |
| 219295_s_at | PCOLCE2 | procollagen C- | 9.91 | 7.76 |
| | | endopeptidase enhancer 2 | | |
| 204712_at | WIF1 | WNT inhibitory factor 1 | 6.92 | 8.45 |
| 227265_at | FGL2 | fibrinogen-like 2 | 5.43 | 9.16 |
| 213661_at | PAMR1 | peptidase domain | 6.02 | 9.7 |
| | | containing associated with | | |
| | | muscle regeneration 1 | | |
| 233180_at | | | 8.03 | 10.07 |
| 202037_s_at | SFRP1 | secreted frizzled-related | 8.65 | 10.49 |
| | | protein 1 | | |
| 201645_at | TNC | tenascin C | 6.73 | 10.78 |
| 235182_at | ISM1 | isthmin 1, angiogenesis | 10.94 | 11.94 |
| | | inhibitor | | |
| 205475_at | SCRG1 | stimulator of | 6.68 | 12.42 |
| | | chondrogenesis 1 | | |
| 243541_at | IL31RA | interleukin 31 receptor A | 6.04 | 13.31 |
| 204455_at | DST | dystonin | 5.36 | 14.23 |
| 237169_at | TNC | tenascin C | 6.33 | 15.33 |
| 227758_at | RERG | RAS-like, estrogen- | 7.65 | 16.58 |
| | | regulated, growth inhibitor | | |
| 202746_at | ITM2A | integral membrane protein | 6.75 | 16.67 |
| | | 2A | | |
| 221898_at | PDPN | podoplanin | 9.83 | 18.49 |
| 219525_at | SLC47A1 | solute carrier family 47 | 6.91 | 21.81 |
| | | (multidrug and toxin | | |
| | | extrusion), member 1 | | |
| 202291_s_at | MGP | matrix Gla protein | 22.76 | 23.65 |
| 213880_at | LGR5 | leucine-rich repeat | 5.32 | 23.8 |
| | | containing G protein- | | |
| | | coupled receptor 5 | | |
| 212667_at | SPARC | secreted protein, acidic, | 11.38 | 25.99 |
| | | cysteine-rich (osteonectin) | | |
| 225911_at | NPNT | nephronectin | 6.18 | 43.77 |
| 227370_at | FAM171B | family with sequence | 5.86 | 14.6 |
| | | similarity 171, member B | | |
| 221127_s_at | DKK 3 | dickkopf WNT signaling | 8.51 | 6.46 |
| | | pathway inhibitor 3 | | |

TABLE 2
Downregulated genes in actively miniaturizing/not currently miniaturizing
but likely to miniaturize vs. non-miniaturizing hair follicles.

| | | | | Likely to |
|---|---|---|---|---|
| | | | Miniaturizing | miniaturize |
| | | | vs. non- | vs. non- |
| | Gene | | miniaturizing | miniaturizing |
| Transcript ID | Symbol | Gene Name | (Fold Change) | (Fold Change) |
| 211819_s_at | SORBS1 | sorbin and SH3 domain | −9.6 | −84.4 |
| | | containing 1 | | |
| 1556409_a_at | LOC105369434 | uncharacterized | −5.51 | −65.33 |
| | | LOC105369434 | | |
| 1552960_at | LRRC15 | leucine rich repeat | −5.46 | −60.01 |
| | | containing 15 | | |
| 221416_at | PLA2G2F | phospholipase A2, | −7.24 | −47.85 |
| | | group IIF | | |
| 214414_x_at | HBA1; HBA2 | hemoglobin, alpha 1; | −6.74 | −33.65 |
| | | hemoglobin, alpha 2 | | |
| 1554766_s_at | | | −5.19 | −31.82 |
| 1559021_at | C3orf52 | chromosome 3 open | −6.16 | −23.19 |
| | | reading frame 52 | | |
| 244799_s_at | | | −7.14 | −22.71 |
| 202885_s_at | PPP2R1B | protein phosphatase 2, | −5.7 | −22.53 |
| | | regulatory subunit A, | | |
| | | beta | | |
| 1553211_at | ANKFN1 | ankyrin-repeat and | −6.58 | −15.07 |
| | | fibronectin type III | | |
| | | domain containing 1 | | |
| 238636_at | CACNA1C | calcium channel, | −9.65 | −15.05 |
| | | voltage-dependent, L | | |
| | | type, alpha 1C subunit | | |
| 229103_at | LOC101929777; | uncharacterized | −6.64 | −14.63 |
| | WNT3 | LOC101929777; | | |
| | | wingless-type MMTV | | |
| | | integration site family, | | |
| | | member 3 | | |
| 1553882_at | LINC00504 | long intergenic non- | −5.54 | −14.57 |
| | | protein coding RNA 504 | | |
| 220148_at | ALDH8A1 | aldehyde | −10.59 | −13.29 |
| | | dehydrogenase 8 family, | | |
| | | member A1 | | |
| 1553388_at | FAM26D | family with sequence | −5.22 | −12.33 |
| | | similarity 26, member D | | |
| 205268_s_at | ADD2 | adducin 2 (beta) | −6.35 | −10.8 |
| 204420_at | FOSL1 | FOS-like antigen 1 | −5.21 | −10.38 |
| 223775_at | HHIP | hedgehog interacting | −5.76 | −9.68 |
| | | protein | | |
| 1554646_at | OSBPL1A | oxysterol binding | −5.09 | −8.23 |
| | | protein-like 1A | | |
| 218980_at | FHOD3 | formin homology 2 | −6.55 | −8.02 |
| | | domain containing 3 | | |
| 1556037_s_at | HHIP | hedgehog interacting | −6.65 | −7.96 |
| | | protein | | |
| 208250_s_at | DMBT1 | deleted in malignant | −6.34 | −7.67 |
| | | brain tumors 1 | | |
| 1555774_at | ZAR1 | zygote arrest 1 | −5.64 | −6.93 |
| 234895_at | CTLA4 | cytotoxic T-lymphocyte- | −6.51 | −6.9 |
| | | associated protein 4 | | |
| 203130_s_at | KIF5C | kinesin family member | −5.01 | −6.68 |
| | | 5C | | |
| 227736_at | C10orf99 | chromosome 10 open | −6.54 | −5.29 |
| | | reading frame 99 | | |

TABLE 3
Upregulated genes in actively miniaturizing vs not yet
miniaturizing but likely to miniaturize hair follicles.

| | | | | Likely to |
|---|---|---|---|---|
| | | | Miniaturizing | miniaturize |
| | | | vs. non- | vs. non- |
| | Gene | | miniaturizing | miniaturizing |
| Transcript ID | Symbol | Gene Name | (Fold Change) | (Fold Change) |
| 1553721_at | RNF152 | ring finger protein 152 | 5.54 | −8.59 |
| 229853_at | ANOS1 | anosmin 1 | 5.22 | −3.17 |
| 214680_at | NTRK2 | neurotrophic tyrosine | 9.26 | −2.14 |
| | | kinase, receptor, type 2 | | |
| 236173_s_at | LRIG1 | leucine-rich repeats and | 6.74 | −2 |
| | | immunoglobulin-like | | |
| | | domains 1 | | |
| 205920_at | SLC6A6 | solute carrier family 6 | 5.33 | −1.89 |
| | | (neurotransmitter | | |
| | | transporter), member 6 | | |
| 204636_at | COL17A1 | collagen, type XVII, alpha 1 | 5.91 | −1.82 |
| 217202_s_at | GLUL | glutamate-ammonia ligase | 9.64 | −1.65 |
| 219872_at | FAM198B | family with sequence | 5.05 | −1.55 |
| | | similarity 198, member B | | |
| 1558019_at | | | 5.66 | −1.44 |
| 211215_x_at | DIO2 | deiodinase, iodothyronine, | 6.79 | −1.28 |
| | | type II | | |
| 1565892_at | | | 7.65 | −1.27 |
| 242762_s_at | FAM171B | family with sequence | 8.98 | −1.14 |
| | | similarity 171, member B | | |
| 237177_at | CNTN4 | contactin 4 | 6.14 | 1.03 |
| 232098_at | DST | dystonin | 9.66 | 1.85 |
| 228858_at | | | 7.77 | 1.58 |
| 220406_at | TGFB2 | transforming growth factor | 5.64 | −1.22 |
| | | beta 2 | | |
| 203699_s_at | DIO2 | deiodinase, iodothyronine, | 8.32 | −1.67 |
| | | type II | | |
| 238022_at | CRNDE | colorectal neoplasia | 5.25 | 2.62 |
| | | differentially expressed | | |
| | | (non-protein coding) | | |
| 202036_s_at | SFRP1 | secreted frizzled-related | 8.09 | −1.58 |
| | | protein 1 | | |

1. An ex vivo method of determining the phenotype of a hair follicle comprising determining the expression profile of cellular material attached to a hair shaft plucked from said hair follicle.

2. The method according to claim 1, wherein the phenotype is androgen sensitivity.

3. The method according to claim 1, wherein the expression profile is:
(a) a transcriptome or gene expression profile; or
(b) a protein expression profile.

4. The method according to claim 3, wherein said expression profile is a transcriptome or gene expression profile determined by RT-qPCR, northern blotting, DNA/RNA microarray and/or RNA-Seq.

5. The method according to claim 1, wherein the expression profile includes the expression of one or more, preferably two or more, more preferably five or more of: NPNT, SPARC, LGR5, MGP, SLC47A1, PDPN, ITM2A, RERG, TNC, FAM171B, SORBS1, LOC105369434, LRRC15, PLA2G2F, HBA1, HBA2, C3orf52, PPP2R1B, ANKFN1, CACNA1C, LOC101929777 (WNT3), DST, GLUL, NTRK2, DIO2, SFRP1, LRIG1, CNTN4, COL17A1, TGFB2, IL31RA, SCRG1, ISM1, PAMR1, FGL2, WIF1, PCOLCE2, CDH11, DKK3, GAS1, FGF18, P2RY1, LINC00504, ALDH8A1, FAM26D, ADD2, FOSL1, HHIP, OSBPL1A, FHOD3, DMBT1, ZAR1, CTLA4, KIF5C, C10orf99, RNF152, SLC6A6, CRNDE, ANOS1 and FAM198B, or any combination thereof.

6. The method according to claim 5, wherein:
a) expression or up-regulation of one or more of NPNT, SPARC, LGR5, MGP, SLC47A1, PDPN, ITM2A, RERG, TNC, FAM171B, DST, IL31RA, SCRG1, ISM1, SFRP1, PAMR1, FLG2, WIF1, PCOLCE2, CDH11, NTRK2, DKK3, GAS1, FGF18, DKK3 and/or P2RY1, or any combination thereof is associated with androgen sensitivity; and/or
b) lack of expression or down-regulation of one or more of SORBS1, LOC105369434, LRRC15, PLA2G2F, HBA1, HBA2, C3orf52, PPP2R1B, ANKFN1, CACNA1C, LOC101929777 (WNT3), LINC00504, ALDH8A1, FAM26D, ADD2, FOSL1, HHIP, OSBPL1A, FHOD3, DMBT1, ZAR1, CTLA4, KIF5C and/or C10orf99, or any combination thereof is associated with androgen sensitivity; and/or
c) expression or up-regulation of one or more of DST, GLUL, NTRK2, FAM171B, DIO2, SFRP1, LRIG1, CNTN4, COL17A1, TGFB2, RNF152, SLC6A6, CRNDE, ANOS1 and/or FAM198B, or any combination thereof is associated with androgen sensitivity.

7. The method according to claim 5, wherein:
a) expression or up-regulation of one or more of NPNT, SPARC, LGR5, MGP, SLC47A1, PDPN, ITM2A, RERG, TNC, and/or FAM171B, or any combination thereof is associated with androgen sensitivity;
b) lack of expression or down-regulation of one or more of SORBS1, LOC105369434, LRRC15, PLA2G2F, HBA1, HBA2, C3orf52, PPP2R1B, ANKFN1, CACNAI1 and/or LOC101929777 (WNT3), or any combination thereof is associated with androgen sensitivity; and/or
c) expression or up-regulation of one or more of DST, GLUL, NTRK2, FAM171B, DIO2, SFRP1, LRIG1, CNTN4, COL17A1, and/or TGFB2, or any combination thereof is associated with androgen sensitivity.

8. A method for assessing the extent of potential future hair loss in an individual, said method comprising carrying out a method as defined in claim 1 on hair shafts plucked from a plurality of hair follicles.

9. The method according to claim 8, wherein the boundary between androgen-sensitive and androgen-insensitive regions of an individual's scalp is identified, wherein:
a) carrying out a the method is performed on hair shafts plucked from one or more hair follicles from a region of the scalp of said individual; further comprising:
b) determining the sensitivity of said region to an androgen, wherein said region is androgen sensitive if said one or more hair follicles are androgen sensitive;
c) repeating steps (a) and (b) one or more times on hair shafts plucked from one or more hair follicles from at least one different region of the scalp of said individual;
d) generating a map of the androgen-sensitive and androgen-insensitive hair follicles within said regions of the scalp to identify the boundary between androgen-sensitive and androgen-insensitive regions of an individual's scalp.

10. A method for determining a region comprising hair follicles suitable:
(a) for hair transplantation in an individual; and/or
(b) as a source of cells for use in a method of rejuvenating hair follicles in an individual;
wherein said method comprises:
(i) carrying out a method as defined in claim 1 on hair shafts plucked from one or more hair follicles from a region of the scalp of said individual;
(ii) determining the sensitivity of said region to an androgen, wherein said region is androgen sensitive if said one or more hair follicles are androgen sensitive;
(iii) repeating steps (a) and (b) one or more times on hair shafts plucked from one or more hair follicles from at least one different region of the scalp of said individual;
(iv) generating a map of the androgen-sensitive and androgen-insensitive hair follicles within said regions of the scalp to identify the boundary between androgen-sensitive and androgen-insensitive regions of an individual's scalp;
wherein an area comprising hair follicles suitable for hair transplantation and/or as a source of cells for use in a method of rejuvenating hair follicles comprises one or more androgen-insensitive hair follicle.

11. The method according to claim 8, which is carried out before hair loss in said individual reaches visible levels, and preferably before hair loss commences.

12. A method for assessing the outcome of a previous hair follicle rejuvenation treatment, said method comprising carrying out a method as defined in claim 1 on hair shafts plucked from one or more hair follicles from a region previously containing androgen sensitive hair follicles and subjected to hair follicle rejuvenation; wherein the outcome is positive if there is a change in the expression profile of the cellular material attached to hair shafts plucked from said one or more hair follicles.

13. The method according to claim 12, wherein the change in the expression profile of the cellular material attached to the hair shafts plucked from said one or more hair follicles is a change from an expression profile associated with androgen-sensitivity to an expression profile associated with androgen-insensitivity.

14. The method according to claim 12, wherein an expression profile associated with androgen-insensitivity comprises:
a) lack of expression or down-regulation of one or more of NPNT, SPARC, LGR5, MGP, SLC47A1, PDPN, ITM2A, RERG, TNC, and/or FAM171B, or any combination thereof;
b) expression or up-regulation of one or more of SORBS1, LOC105369434, LRRC15, PLA2G2F, HBA1, HBA2, C3orf52, PPP2R1B, ANKFN1, CACNAI1 and/or LOC101929777 (WNT3), or any combination thereof; and/or
c) lack of expression or down-regulation of one or more of DST, GLUL, NTRK2, FAM171B, DIO2, SFRP1, LRIG1, CNTN4, COL17A1, and/or TGFB2, or any combination thereof.

15. A method for determining whether a hair follicle is miniaturizing and suitable for a hair rejuvenation treatment, said method comprising carrying out a method as defined in claim 1, to determine the androgen-sensitivity of the hair follicle.

16. The method according to claim 15, wherein an expression profile associated with androgen-sensitivity comprises:
a) expression or up-regulation of one or more of NPNT, SPARC, LGR5, MGP, SLC47A1, PDPN, ITM2A, RERG, TNC, and/or FAM171B, or any combination thereof;
b) lack of expression or down-regulation of one or more of SORBS1, LOC105369434, LRRC15, PLA2G2F, HBA1, HBA2, C3orf52, PPP2R1B, ANKFN1, CACNAI1 and/or LOC101929777 (WNT3), or any combination thereof; and/or
c) expression or up-regulation of one or more of DST, GLUL, NTRK2, FAM171B, DIO2, SFRP1, LRIG1, CNTN4, COL17A1, and/or TGFB2, or any combination thereof.

17. The method according to claim 8, wherein said hair loss is due to androgenetic alopecia or the individual has androgenetic alopecia.

18. The method according to claim 1, wherein said androgen is dihydrotestosterone (DHT).

19. The method according to anyone of claim 6, wherein said upregulation and/or downregulation is compared with the corresponding expression profile of cellular material attached to a hair shaft plucked from an androgen-insensitive hair follicle.

20. The method according to claim 19, wherein the hair shaft plucked from an androgen-insensitive hair follicle is a hair shaft from the temporal or occipital region of the scalp, preferably a terminal hair shaft from the temporal region.

21. The method according to claim 14, wherein said upregulation and/or downregulation is compared with the corresponding expression profile of cellular material attached to a hair shaft plucked from an androgen-sensitive hair follicle.

22. The method according to claim 21, wherein the hair shaft plucked from an androgen-sensitive hair follicle is a hair shaft from the frontal or vertex region of the scalp, preferably a minimizing or vellus hair shaft from the frontal region.

23. The method according to claim 1, wherein said individual is a human and preferably the hair follicle is from the scalp.

24. A kit for collecting hair shafts for use in an ex vivo method of determining the androgen sensitivity of a plurality of hair follicles, the kit comprising:
a flexible cap defining:
(a) a receiving portion for accommodating a wearer's head, and
(b) a plurality of apertures through the flexible cap, the apertures distributed around the receiving portion and configured to allow a plurality of hair shafts from different regions of the wearer's scalp to be passed from the interior of the cap, through the aperture, to the exterior of the cap to allow hair shafts to be plucked from the hair follicles; and
a plurality of containers, wherein each container is labelled to correspond to a respective aperture of the plurality of apertures, for containing the plucked hair shafts from the different regions of the wearer's scalp.

25. The kit of claim 24 wherein each aperture is individually labelled with the same label as the corresponding labelled container.

26. The kit of claim 24 wherein the flexible cap comprises markings for positioning the flexible cap with respect to the wearer's head such that each of the plurality of apertures are positioned over corresponding selected portion of the wearer's scalp for obtaining hair shafts from hair follicles from selected regions of the wearer's scalp.
